(19) United States
(12) Patent Application Publication
(10) Pub. No.: US 2023/0098149 A1
(43) Pub. Date: Mar. 30, 2023

(54) METHODS FOR DETECTING NEUTRALIZING ANTIBODIES

(71) Applicant: Duke University, Durham, NC (US)

(72) Inventors: Ashutosh Chilkoti, Durham, NC (US); Jacob Heggestad, Durham, NC (US); David Kinnamon, Durham, NC (US); Angus Hucknall, Durham, NC (US)

(21) Appl. No.: 17/957,373

(22) Filed: Sep. 30, 2022

Related U.S. Application Data
(60) Provisional application No. 63/250,813, filed on Sep. 30, 2021.

(51) Int. Cl. G01N 33/569; G01N 21/64

(52) U.S. Cl. CPC G01N 33/56983; G01N 21/6428; G01N 2021/6439; G01N 2333/165; G01N 2469/20

(57) ABSTRACT

Disclosed herein are devices that can be used to detect neutralizing antibodies against multiple pathogens in a quick and accurate manner. An example device includes a substrate; a non-fouling layer; a plurality of pathogen regions, each pathogen region including a different pathogen; and at least one detection region, the detection region including a detection agent that is capable of specifically binding each pathogen and an excipient. In addition, an example method includes contacting a biological sample with a device, and detecting the presence of a neutralizing antibody in the biological sample for each pathogen, wherein the presence of the neutralizing antibody is detected by inhibiting the binding of the detection agent to each pathogen.

### CROSS-REFERENCE TO RELATED APPLICATIONS

This application claims priority to U.S. Provisional Patent Application No. 63/250,813 filed on Sep. 30, 2021, which is incorporated fully herein by reference.

### STATEMENT REGARDING FEDERALLY SPONSORED RESEARCH

This invention was made with government support under Grant Nos. K08HL130557, R01 AI159992, P30-CA014236, and UC6AI058607 awarded by the National Institutes of Health; Grant No. CBET2029361 awarded by the National Science Foundation; and Grant No. HR0011-17-2-0069 awarded by the Department of Defense & Defense Advanced Research Projects Agency. The government has certain rights in the invention.

### TECHNICAL FIELD

The present disclosure relates to devices and methods for detecting neutralizing antibodies in a biological sample.

### INTRODUCTION

Assays to detect anti-SARS-CoV-2 antibodies are a useful tool to assess natural or vaccine-induced humoral response at the individual patient level and for epidemiological surveillance at the population level. While many antibody binding assays have been developed for COVID-19 serodiagnosis, these tests are unable to determine the specific fraction of antibodies that can potentially neutralize the SARS-CoV-2 virus and thus confer protection.

### SUMMARY

In one aspect, disclosed herein are devices that include a substrate a non-fouling layer positioned on the substrate; the non-fouling layer including a brush-like polymer; a plurality of pathogen regions positioned on the non-fouling layer, each pathogen region including a different pathogen; and at least one detection region positioned on the non-fouling layer spatially separated from the pathogen regions, the detection region including a detection agent and an excipient, wherein the detection agent solubilizes upon contacting a biological sample and is capable of specifically binding to each pathogen.

In another aspect, disclosed herein are methods of detecting a neutralizing antibody, the method including contacting a biological sample with a disclosed device; and detecting the presence of a neutralizing antibody in the biological sample for each pathogen, wherein the presence of the neutralizing antibody is detected by inhibiting the binding of the detection agent to each pathogen.

In another aspect, disclosed herein are methods of determining a neutralizing activity of a vaccine, the method including obtaining a biological sample from a subject that has received a vaccine; contacting a biological sample with a device as disclosed herein; and detecting the presence of a neutralizing antibody induced by the vaccine for each pathogen, wherein the presence of the neutralizing antibody is detected by inhibiting the binding of the detection agent to each pathogen.

In another aspect, disclosed herein are methods of determining a neutralizing activity of a subject exposed to a pathogen but not having received a vaccine against the pathogen, the method including obtaining a biological sample from a subject that has been exposed to a pathogen; contacting the biological sample with a device as disclosed herein; and detecting the presence of a neutralizing antibody induced by the subject's immune system for each pathogen on the device, wherein the presence of the neutralizing antibody is detected by inhibiting the binding of the detection agent to each pathogen.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows a schematic of an example assay. (FIG. 1A) WT, B.1.1.7, P.1, and B.1.351 receptor binding domain (RBD) are inkjet printed onto a poly(oligoethylene glycol methyl ether methacrylate) (POEGMA) surface. Nearby, fluorescently labeled angiotensin-converting enzyme-2 (ACE2) is inkjet printed onto a dissolvable trehalose pad. When a plasma or serum sample is added to a chip, the trehalose pad dissolves, liberating the ACE2 from the surface, which diffuses across the surface. If a sample does not contain neutralizing antibodies (nAbs), ACE2 binds to an RBD, leading to an increased fluorescence signal. If a sample contains nAbs, the nAbs block the ACE2-RBD binding interaction, leading to a lower fluorescence signal. (FIG. 1B, upper panel) SARS-CoV-2 variant RBD mutations. Variant B.1.1.7 contains one RBD mutation: N501Y. Variants P.1 and B.1.351 each contain three RBD mutations: K417T (P.1)/K417N (B.1.351), E484K, and N501Y. (FIG. 1B, lower panel) nAbs interfere with the RBD-ACE2 binding interaction to varying degrees. Greater blocking of this interaction indicates greater antibody neutralization.

FIG. 2 shows an example assay to assess potency of patient-derived, recombinant, and EUA-approved therapeutic mAbs. (FIG. 2A) Percentage of RBD-ACE2 binding blocked by 12 convalescent patient-derived mAbs against four RBD variants (WT, B.1.1.7, P.1, and B.1.351) as measured by the CoVariant-SCAN. Each mAb was spiked into pre-pandemic pooled human serum (PHS), and a 7-point dilution series (30 μg/mL high dose, 1:3 dilutions) was tested in duplicate. Each dose was incubated for 1 h and was then imaged on a GenePix scanner. Furthest left data points are blanks. (FIG. 2B) Percentage of RBD-ACE2 binding blocked by 6 commercially purchased recombinant nAbs. 7-point dilution series tested in the same fashion as (FIG. 2A) but in triplicate. (FIG. 2C) Percentage of RBD-ACE2 binding blocked by Regeneron® therapeutic antibodies. Antibodies were tested individually (REGN10933 & REGN10987) and together in the therapeutic cocktail (REGEN-CoV). Each data point represents the average of three independent assays. (FIG. 2D) Percentage of binding blocked at the highest dose (30 μg/mL) for each variant (left) and log-transformed inhibitory concentration that blocks>20% binding for each variant (right). Antibodies that do not block>20% of ACE2 binding are marked with an “X”. For both definitions, more potent antibodies are darker red. Antibodies that block all variants similarly are outlined with red dashes.

FIG. 3 shows an example assay to assess natural humoral immunity. (FIG. 3A) Plasma samples were collected from four cohorts: pre-pandemic COVID-19 (−) individuals, mildly symptomatic COVID-19 (+) patients, hospitalized COVID-19 (+) patients not requiring ICU admission, and COVID-19 (+) patients admitted to the ICU. The table provides information on the number of samples and time of collection. (FIG. 3B) Percent blocking of RBD-ACE2 binding by patient plasma as measured by CoVariant-SCAN. Data is divided by patient cohort, where lines connect data from the same patient. Asterisks indicate significant difference from WT (* indicates adjusted p<0.05, ** indicates adjusted p<0.01, *** indicates adjusted p<0.001, **** indicates adjusted p<0.0001) based on Dunnett's multiple comparisons test. (FIG. 3C) Same dataset split by RBD variant (PP=pre-pandemic, Hos=hospitalized). Asterisks indicate a significant difference in percentage blocked between the marked cohorts using one-way ANOVA and Tukey's multiple comparison post-hoc test. All points shown are the average of two replicates.

FIG. 4 shows an example assay to assess vaccine-induced humoral immunity. (FIG. 4A) Plasma samples were collected from three cohorts: pre-pandemic (PP) healthy controls, individuals who received the Pfizer-BioNTech vaccine (BNT162b2 mRNA), and individuals who received the Moderna vaccine (SARS-Co-2 mRNA-1273). The data table provides information on the number of samples and time of collection. (FIG. 4B) Longitudinal data from two individuals who received the Pfizer vaccine (901 and 907) and two individuals who received the Moderna vaccine (902 and 903). Plasma samples from prior to dose one, after dose one, and after dose two were assayed on the CoVariant-SCAN in duplicate (SEM shown). (FIG. 4C) Percent blocking of RBD-ACE2 binding by patient plasma as measured by CoVariant-SCAN. Data is divided by vaccine cohort, where lines connect data from the same patient. Asterisks indicate significant difference from WT (** indicates adjusted p<0.01) based on Dunnett's multiple comparisons test. All points shown are the average of two replicates. Samples 10 and 3 in panel E were tested in an indirect assay to determine if anti-RBD binding antibodies were present, despite low blocking activity. (FIG. 4D) Same dataset as FIG. 4C split by variant. For each variant there was a significant difference in ACE2 blocking compared to pre-pandemic negative controls (**** indicates adjusted p<0.0001) based on Tukey's multiple comparisons test. There was no significant difference between the vaccine types for any RBD variant.

FIG. 5 shows an example assay including B.1.617.2 (Delta) variants of concern (VOC). (FIG. 5A) The RBD protein for B.1.617.2, which includes two mutations—L452R and T478K—was incorporated into the CoVariant-SCAN assay. (FIG. 5B) Percentage of RBD-ACE2 binding blocked by Regeneron® therapeutic antibodies. Antibodies were tested individually (REGN10933 & REGN10987) and together in the therapeutic cocktail (REGEN-CoV). Each data point represents the average of three independent assays. (FIG. 5C) Percent blocking of RBD-ACE2 binding by Pfizer (n=12), Moderna (n=4), and Johnson & Johnson (n=3) vaccinee plasma as measured by the CoVariant-SCAN. Asterisks indicate significant difference from WT (* indicates adjusted p<0.05, ** indicates adjusted p<0.01) based on Dunnett's multiple comparisons test. All points shown are the average of two replicates. Dashed lines indicate that an individual had a previous confirmed COVID-19 diagnosis.

FIG. 6 shows an example assay print layout. Shown is the architecture of CoVariant-SCAN assays used in the Examples. Each standard glass microscope slide contains 24 individual assays. Also shown is a zoomed in view of one individual assay. All measurements listed are in mm.

FIG. 7 shows the impact of incubation time in an example assay. A 7-point dose-response curve for mAb 40592-R001 at a starting concentration of 30 μg/mL using WT RBD as the pathogen at four different incubation times: 20 min, 40 min, 60 min, and 120 min. The log-transformed fluorescent intensity at each dose is plotted against mAb concentration.

### DETAILED DESCRIPTION

With more transmissible and virulent pathogen strains circulating globally, there is an urgent need for a test that can measure nAbs against several VOCs simultaneously by an easily deployable rapid test. Such a test could be useful to study the impact of RBD mutations on neutralization, to monitor the efficacy of vaccines against circulating VOCs in low resource settings, to identify individuals who may be susceptible to re-infection or breakthrough infections even after vaccination, and to identify patients who may benefit from (monoclonal antibody) mAb therapies.

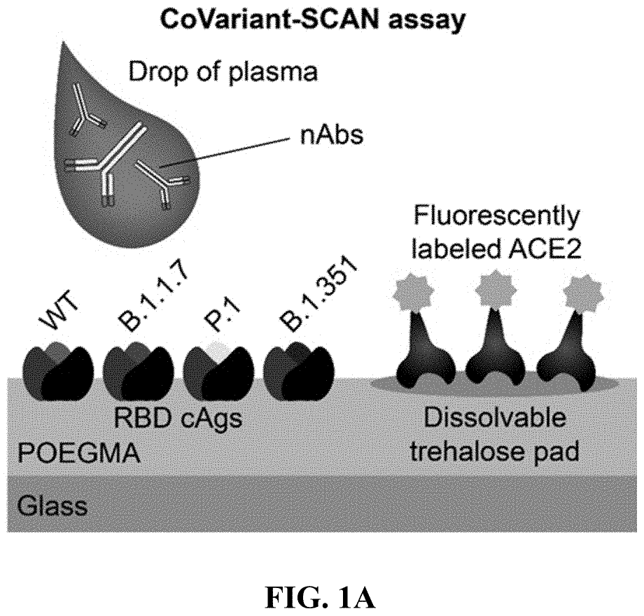

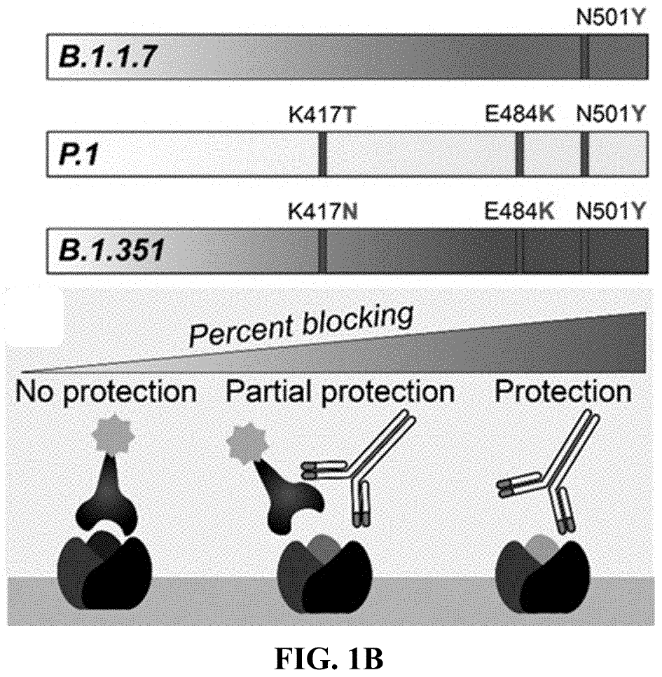

To address this need and the deficiencies of current detection methods, disclosed herein is a rapid test, termed the CoVariant-SCAN (Covid-19 Variant Spike-ACE2 Competitive Antibody Neutralization) assay that can evaluate the ability of host nAbs to block the pathologic interaction between variants of viral RBD and human ACE2 within 1 h from a drop of plasma (FIG. 1A). However, the disclosed rapid test can be applied to any suitable pathological interaction. As proof-of-principle, the performance of the disclosed assay was demonstrated against four SARS-CoV-2 strains—wild type (WT), B.1.1.7, P.1, and B.1.351. This assay was constructed by inkjet printing RBD proteins from each variant (FIG. 1B, upper panel) onto a “non-fouling” poly(oligoethylene glycol methyl ether methacrylate) (POEGMA) coating. Nearby, fluorescently labeled human ACE2 was inkjet printed upon a dissolvable trehalose pad. When a sample without nAbs is added, fluorescently labeled ACE2 dissolves from the POEMGA brush and binds to RBD capture sites, leading to a high fluorescence signal. In the presence of potential nAbs, the RBD-ACE2 interaction can be partially or completely blocked, resulting in a decrease in fluorescence signal (FIG. 1B, lower panel). Importantly, the multiplexing capability of CoVariant-SCAN was demonstrated by simultaneously assessing the neutralizing activity against WT, B.1.1.7, P.1 and B.1.351 from a single sample. The CoVariant-SCAN was used to assess the efficacy of known neutralizing therapeutic mAbs, natural immunity from convalescent plasma and vaccine-induced immunity.

There are at least several potential scenarios where the disclosed devices and methods thereof could be useful. First, it could be deployed as an epidemiological tool to assess the efficacy of vaccines against circulating or emerging VOCs in specific regions. Second, it could be used to monitor individual patients' risk for future infection by a circulating VOC based on their nAb profile. Similarly, the CoVariant-SCAN could be used at the bedside to test patients presenting with acute COVID-19 who are either known to have been infected by a VOC or if there is a high burden of VOC in their community making it likely that their infection is caused by a VOC. Patients with low neutralizing activity could be treated immediately with the mAb cocktail therapy to reduce the likelihood of severe infection. This approach could be useful for patients who are immunocompromised at the time of a viral infection or vaccination, as they are likely to have a weaker humoral response and therefore are more at risk for re-infection and/or severe disease.

### 1. DEFINITIONS

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The terms “biological sample” or “sample,” as used herein, refer to any material that is taken from its native or natural state, so as to facilitate any desirable manipulation or further processing and/or modification. A sample or a biological sample can include a cell, a tissue, a fluid (e.g., a biological fluid), a protein (e.g., antibody, enzyme, soluble protein, insoluble protein), a polynucleotide (e.g., RNA, DNA), a membrane preparation, and the like, that can optionally be further isolated and/or purified from its native or natural state. Example biological samples include, but are not limited to, blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, and amniotic fluid. A biological sample may be in its natural state or in a modified state by the addition of components such as reagents, or removal of one or more natural constituents (e.g., blood plasma).

The term “detection moiety,” as used herein, refers to a moiety or compound that is detectable by methods including, but not limited to, spectroscopic, photochemical, biochemical, immunochemical, chemical, electrochemical, radioactivity, and other physical means. A detection moiety may be detectable directly or indirectly. A non-limiting example of an indirectly detectable detection moiety is biotin, which may bind to avidin or streptavidin comprising a detection moiety such as a fluorophore. Example detection moieties include, but are not limited to, fluorophores, chromophores, radiolabels, polynucleotides, small molecules, enzymes, nanoparticles, and upconverters.

The terms “inhibit,” “inhibition,” or “inhibiting,” as used herein, refer to the reduction or suppression of a given biological process, condition, symptom, disorder, or disease, or a significant decrease in the baseline activity of a biological activity or process.

The term “neutralizing antibody,” as used herein, refers to an antibody that can defend a host (e.g., subject) from a pathogen by neutralizing and/or inhibiting the biological effect of the pathogen. For example, a neutralizing antibody can inhibit or limit the binding of a pathogen to its pathological binding partner (e.g., cellular receptor). By binding specifically to a pathogen, a neutralizing antibody can prevent or inhibit the pathogen from interacting with its host cells. Neutralizing antibodies are part of the humoral response of the adaptive immune system against pathogens, such as viruses, intracellular bacteria and microbial toxins, and are secreted by adaptive immune response cells as soluble proteins.

The term “pathogen,” as used herein, refers to any microorganism or fragment thereof capable of inducing a disease in a subject. Examples include, but are not limited to, a bacterium, a fungus, a virus, a parasite, or a fragment thereof. The pathogen may include a whole pathogen cell, or a part of the pathogen cell, e.g., a cell wall component, an associated protein etc. Pathogens suitable for use in the disclosed devices and methods thereof can be derived from a subject, an in vitro culture, a microorganism lysate, a crude lysate, or a purified lysate, or alternatively, the pathogen may be a synthetic pathogen (e.g., expressed recombinantly).

The term “pathological binding pair,” as used herein, refers to two molecules that exhibit specific binding to one another, or increased binding to one another relative to other molecules, and that participate together in a pathological interaction. A pathological interaction is one where a pathogen binds to another molecule to facilitate the spread of the pathogen in the host or host cell. An example molecule that a pathogen can specifically bind to in a pathological interaction is an extracellular receptor. Accordingly, an example pathological binding pair includes a pathogen and an extracellular receptor or protein, glycan, or lipid involved in pathogen entry and/or replication. Typically, for the disclosed devices and methods thereof one member of the binding pair is the pathogen, and the detection agent may serve as the second member of the pathological binding pair.

A “protein” or “polypeptide” is a linked sequence of 50 or more amino acids linked by peptide bonds. A peptide is a linked sequence of 2 to 50 amino acids linked by peptide bonds. The polypeptide and peptide can be natural, synthetic, or a modification or combination of natural and synthetic. Proteins and polypeptides include proteins such as binding proteins, receptors, and antibodies. The terms “polypeptide,” and “protein” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tall domains, “Domains” are portions of a polypeptide that form a compact unit of the polypeptide and are typically 15 to 350 amino acids long. Example domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length, in some embodiments, a motif includes 3, 4, 5, 6, or 7 sequential amino acids. A domain may be comprised of a series of motifs, which may be similar or different.

The term “region,” as used herein, refers to a defined area on the surface of a material. A region can be identified and bounded by a distinct interface between two materials having different compositions.

The term “subject,” as used herein, refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. The subject may also be referred to as a host.

The term “vaccine,” as used herein, includes any composition containing an immunogenic determinant which stimulates the immune system such that it can better respond to a subsequent pathogen. A vaccine usually contains an immunogenic determinant, e.g., an antigen, and an adjuvant, the adjuvant serving to non-specifically enhance the immune response to that immunogenic determinant. Currently produced vaccines predominantly activate the humoral immune system, e.g., the antibody dependent immune response. Other vaccines focus on activating the cell-mediated immune system including cytotoxic T lymphocytes which are capable of killing targeted pathogens.

The term “variant,” as used herein, refers to a peptide or protein that differs in amino acid sequence by the insertion, deletion, or conservative substitution of amino acids, but retain at least one biological activity relative to a reference peptide or protein. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids. See Kyte et al., J. Mol. Biol. 1982, 757, 105-132, which is incorporated by reference herein in its entirety. The hydropathic index of an amino acid is based on a consideration of its hydrophobicity and charge. It is known in the art that amino acids of similar hydropathic indexes can be substituted and retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. DEVICES

Disclosed herein are devices that can be used in methods of detecting neutralizing antibodies. The devices can use principles found in competitive inhibition assays but can outperform comparative assays due to the advantageous structure of the disclosed devices. Generally, devices can include a substrate; a non-fouling layer; a plurality of pathogen regions—each pathogen region including a different pathogen; and at least one detection region—the detection region including a detection agent that is capable of specifically binding a pathogen and an excipient. Upon contact with a liquid, such as a biological sample, the excipient can be dissolved and the detection agent can be solubilized, which can allow the detection agent to interact with other aspects of the device, such as the pathogens and regions thereof. Accordingly, the device can take advantage of competing binding interactions between the detection agent and other molecules that are capable of binding the pathogens, such as neutralizing antibodies in a biological sample.

### A. Substrate

The substrate can act as a base of the device and can allow for other layers and/or components to be positioned on its surface. A variety of different substrates can be used in the device and may include any suitable material that allows for the disclosed devices to perform a desired function, e.g., detecting neutralizing antibodies. Examples include, but are not limited to, metals, metal oxides, alloys, semiconductors, polymers (such as organic polymers in any suitable form including woven, nonwoven, molded, extruded, cast, etc.), silicon, silicon oxide, ceramics, glass, and combinations thereof.

Example polymers that can be used to form the substrate include, but are not limited to, poly(ethylene) (PE), poly(propylene) (PP), cis and trans isomers of poly(butadiene) (PB), cis and trans isomers of poly(isoprene), poly(ethylene terephthalate) (PET), polystyrene (PS), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), polyacrylamide and its homologs such as poly(N-isopropyl acrylamide), fluorinated polyacrylate (PFOA), poly(ethylene-butylene) (PEB), polystyrene-acrylonitrile) (SAN), polytetrafluoroethylene (PTFE) and its derivatives, polyolefin plastomers, and combinations and copolymers thereof.

In some embodiments, the substrate includes a glass, a silicon, a metal oxide, a polymer, or a combination thereof. In some embodiments, the substrate includes a glass, a silicon, a metal oxide, or a polymer. In some embodiments, the substrate includes a glass, a silicon, or a polymer. In some embodiments, the substrate includes a glass. In some embodiments, the substrate is a glass.

### B. Non-Fouling Layer

The device includes a non-fouling layer positioned on the substrate (e.g., on a surface of the substrate). The non-fouling layer can decrease non-specific binding and/or adsorption of non-target analytes to the device. Non-fouling, as used herein with respect to the layer, relates to the inhibition (e.g., reduction or prevention) of growth of an organism as well as to non-specific or adventitious binding interactions between the non-fouling layer and an organism or biomolecule (e.g., cell, protein, nucleotide, etc.).

The non-fouling property of the layer can be instilled through the inclusion of a brush- like polymer. The hydrophilic nature of the brush-like polymer can allow a droplet of, e.g., blood to diffuse across the entire non-fouling layer surface to potentially interact with other areas of the device, such as the pathogen region. Generally, brush-like polymers are formed by the polymerization of monomeric core groups having one or more groups that function to inhibit binding of a biomolecule (e.g., cell, protein, nucleotide, carbohydrate/lipid) coupled thereto. The monomeric core group can be coupled to a protein-resistant head group. In some embodiments, the brush-like polymer includes a monomeric core group and a protein-resistant head group coupled to the monomeric core group.

Brush-like polymers can be synthesized using radical polymerization techniques, such as catalytic chain transfer polymerization, iniferter mediated polymerization (e.g., photoiniferter mediated polymerization), free radical polymerization, stable free radical mediated polymerization (SFRP), atom transfer radical polymerization (ATRP), and reversible addition-fragmentation chain transfer (RAFT) polymerization. For example, free radical polymerization of monomers to form brush-like polymers can be carried out in accordance with known techniques, such as described in U.S. Pat. Nos. 6,423,465; 6,413,587; and 6,649,138; U.S. Patent Application Publication No. US 2003/0108879 A1—all of which are incorporated herein by reference in their entirety, and variations thereof which will be apparent to those skilled in the art. Atom transfer radical polymerization of monomers to form brush-like polymers can also be carried out in accordance with known techniques, such as described in U.S. Pat. Nos. 6,541,580 and 6,512,060; U.S. Patent Application Publication No. US 2003/0185741 A1—all of which are incorporated herein by reference in their entirety, and variations thereof which will be apparent to those skilled in the art.

Any suitable core vinyl monomer polymerizable by the processes discussed above can be used, including but not limited to styrenes, acrylonitriles, acetates, acrylates, methacrylates, acrylamides, methacrylamides, vinyl alcohols, vinyl acids, and combinations thereof.

Protein resistant groups can be hydrophilic head groups or kosmotropes. Examples include, but are not limited to, oligosaccharides, tri(propyl sulfoxide), hydroxyl, glycerol, phosphorylcholine, tri(sarcosine) (Sarc), N-acetylpiperazine, betaine, carboxybetaine, sulfobetaine, permethylated sorbitol, hexamethylphosphoramide, an intramolecular zwitterion (for example, —CH₂N⁺(CH₃)₂CH₂CH₂CH₂S0₃) (ZW), and mannitol.

Additional examples of kosmotrope protein resistant head groups can include:

—(OCH₂CH₂)₆OH;

### —O(Mannitol);

—C(O)N(CH₃)CH₂(CH(OCH₃))₄CH₂OCH₃;
—N(CH₃)₃⁺Cl⁻/—SO₃⁻Na⁺(1:1);
—N(CH₃)₂+CH₂CH₂SO₃;
—C(O)Pip(NAc) (Pip=piperazinyl);
—N(CH₃)₂+CH₂CO₂;

### —O([Glc-α(1,4)-Glc-β(1)⁻]);

—C(O)(N(CH₃)CH₂C(O))₃N(CH₃)₂;
—N(CH₃)₂⁺CH₂CH₂CH₂SO₃⁻;
—C(O)N(CH₃)CH₂CH₂N(CH₃)P(O)(N(CH₃)₂)₂⁻; or
—S(O)CH₂CH₂CH₂)₃S(O)CH₃.

In some embodiments, a protein resistant head group includes poly(ethylene glycol) (PEG), for example PEG of from 1 to 30 monomeric units, such as 2 to 25 monomeric units, 3 to 20 monomeric units, 4 to 18 monomeric units, or 2 to 15 monomeric units.

In some embodiments, the non-fouling layer is formed by surface-initiated ATRP (SI-ATRP) of oligo(ethylene glycol)methyl methacrylate (OEGMA) to form a poly(OEGMA) (POEGMA) film. In some embodiments, the non-fouling layer includes a functionalized POEGMA film prepared by copolymerization of a methacrylate and methoxy terminated OEGMA. The POEGMA polymer can be formed in a single step. In some embodiments, the non-fouling layer includes POEGMA.

In general, the brush molecules formed by the processes described herein (or other processes either known in the art or which will be apparent to those skilled in the art), can be from 2 or 5 up to 100 or 200 nanometers in length, or more, and can be deposited on the surface portion at a density of from 10, 20, or 40 to up to 100, 200 or 500 milligrams per meter, or more. In some embodiments, the non-fouling layer has a thickness of about 2 nm to about 500 nm, such as about 2 nm to about 400 nm, about 5 nm to about 300 nm, about 10 nm to about 250 nm, about 2 nm to about 200 nm, or about 5 nm to about 200 nm. In some embodiments, the non-fouling layer is deposited on the substrate at a density of about 10 mg/m² to about 600 mg/m², such as about 20 mg/m² to about 500 mg/m², about 10 mg/m² to about 500 mg/m², about 20 mg/m² to about 400 mg/m², or about 10 mg/m² to about 400 mg/m².

Prior to deposition of further components onto the non-fouling layer, the substrate with an optional linking layer and non-fouling layer can be dry or at least macroscopically dry (that is, dry to the touch or dry to visual inspection, but retaining bound water or water of hydration in the polymer layer). For example, to enhance immobilization of a pathogen, the non-fouling layer can suitably retain bound water or water of hydration, but not bulk surface water. If the substrate with the optional linking layer and non-fouling layer has been stored in desiccated form, bound water or water of hydration can be reintroduced by quickly exposing the non-fouling layer to water (e.g., by dipping into water) and subsequently blow-drying the surface (e.g., with a nitrogen or argon jet). Alternatively, bound water or water of hydration can be reintroduced by exposing the non-fouling layer to ambient air for a time sufficient for atmospheric water to bind to the polymer layer.

Further discussion regarding the non-fouling layer, the substrate, the optional linking layer, e.g., between the substrate and the non-fouling layer, and methods of printing molecules onto devices can be found in U.S. Pat. No. 9,482,664, which is incorporated by reference herein in its entirety.

### C. Pathogen Region

The device includes a plurality of pathogen regions positioned on the non-fouling layer. Each pathogen region can include a different pathogen. For example, if two pathogen regions are present, two different pathogens can be present—where an individual pathogen corresponds to each individual region. The device can include any suitable amount of pathogen regions as long as the device is capable of, e.g., detecting neutralizing antibodies. The device can include 2 to 30 pathogen regions (where each pathogen region can correspond to a different pathogen), such as 2 to 25 pathogen regions, 2 to 20 pathogen regions, 2 to 15 pathogen regions, 2 to 10 pathogen regions, 2 to 8 pathogen regions, 2 to 6 pathogen regions, or 2 to 4 pathogen regions. In some embodiments, the device includes greater than 2 pathogen regions, greater than 3 pathogen regions, greater than 4 pathogen regions, greater than 5 pathogen regions, or greater than 10 pathogen regions. In some embodiments, the device includes less than 30 pathogen regions, less than 25 pathogen regions, less than 20 pathogen regions, less than 15 pathogen regions, or less than 10 pathogen regions. Accordingly, the device can include multiple pathogens that can be assessed for each of its interaction with antibodies in a single biological sample.

The pathogen can be any infectious microorganism that can have antibodies generated against it or against a fragment of the pathogen. In addition, the pathogen can be one member of a pathological binding pair. Example pathogens include, but are not limited to, a bacterium, a fungus, a virus, and a parasite, or a fragment of any of the foregoing. In some embodiments, each pathogen includes a bacterium, a fungus, a virus, a parasite, a fragment thereof, or any combination thereof. In some embodiments, each pathogen includes a bacterium, a fungus, a virus, a parasite, or a fragment thereof.

The pathogen can be a virus or a fragment thereof. Example viruses include, but are not limited to, SARS-CoV-2, Ebola, Zika, adeno-associated virus, and other coronaviruses. In some embodiments, each pathogen includes a virus, a fragment of a virus, a viral protein or a variant thereof, or a combination thereof. In some embodiments, each pathogen includes a virus, a viral protein, or a combination thereof. In some embodiments, each pathogen includes a viral protein derived from SARS-CoV-2 or a variant of SARS-CoV-2.

The pathogen can be any fragment or protein associated with a virus that can meditate or participate in a pathological interaction with a host or host cell. For example, the pathogen can be a spike (S) protein, which has a role in fusion, entry, and transmission of a virus. In some embodiments, at least one of the pathogens includes an S protein. In some embodiments, each pathogen includes an S protein derived from SARS-CoV-2 or a variant of SARS-CoV-2. In addition, the pathogen can be a fragment of an S protein, such as a receptor binding domain (RBD). In some embodiments, at least one of the pathogens includes an RBD. In some embodiments, each pathogen includes an RBD. In some embodiments, each pathogen includes an RBD derived from SARS-CoV-2 or a variant of SARS-CoV-2.

Pathogens can be isolated from naturally occurring sources (e.g., from a subject's blood sample) or produced recombinantly. Pathogens can also be purchased from commercial suppliers. For example, RBD proteins can be purchased from Sino Biological and other like suppliers.

The pathogen can be deposited on the non-fouling layer by any suitable technique such as microprinting or microstamping, piezoelectric or other forms of non-contact printing and direct contact quill printing. When the pathogen is printed on to the non-fouling layer, it may be adsorbed onto the non-fouling layer such that it remains bound when the device is exposed to a fluid, such as a biological sample. The brush-like polymer may also provide a protective environment, such that the pathogen remains stable when the device is stored. For example, the brush-like polymer layer may protect the pathogen against degradation, which can allow the device to be stored under ambient conditions.

The pathogen may be printed onto the non-fouling layer to form a pathogen region. The pathogen regions can be arranged in any particular manner and can include any desirable shape or pattern such as, for example, spots (e.g., of any general geometric shape), lines, or other suitable patterns that allow for identification of the pathogen region on the surface of the non-fouling layer and substrate. In some embodiments, a plurality of pathogens can be arranged in a predetermined pattern such that the identity of the pathogen is associated with a specific location on the non-fouling layer. In some embodiments, the pathogen regions are spotted on the non-fouling layer as a row of individual spots. This arrangement may provide independent replicates and may improve robustness of the assay. For example, a microarray containing microspots of varying pathogen and/or pathogen density may allow a broader range of pathogen concentrations to fall within the dynamic range of a given detector, and may thereby eliminate the dilution series of tests usually run of a single sample.

The pathogen regions can be arranged as an array on the non-fouling layer. When an array is formed by the deposition of multiple pathogens at discrete locations on the non-fouling layer, pathogen densities of 1, 3, 5, 10, 100 or up to 1000 pathogen locations per cm² can be made. Modern non-contact arrayers can be used in the deposition step to produce arrays having up to 1,000,000 pathogen locations per cm². For example, using dip-pen nanolithography, arrays with up to 1 billion discrete pathogen locations per cm² can be prepared. In some embodiments, the pathogen is present on the non-fouling layer at about 1 pathogen/cm²to about 1,000,000,000 pathogens/cm², such as about 1 pathogen/cm²to about 1,000,000 pathogens/cm², about 1 pathogen/cm² to about 500,000 pathogens/cm², about 1 pathogen/cm² to about 800,000 pathogens/cm², about 10 pathogens/cm²to about 100,000 pathogens/cm², about 10 pathogens/cm² to about 50,000 pathogens/cm², about 1 pathogen/cm² to about 10,000 pathogens/cm², or about 1 pathogen/cm² to about 1,000 pathogens/cm².

As discussed elsewhere, the specific molecular species at each pathogen region can be different. In addition, the device may include duplicate pathogen regions, e.g., to provide some redundancy or control.

### D. Detection Region

The device includes at least one detection region positioned on the non-fouling layer. In some embodiments, the device includes a plurality of detection regions. The detection region(s) are spatially separated from the pathogen regions on the non-fouling layer. The detection region includes a detection agent and an excipient. The detection agent can be non-covalently bound to the non-fouling layer. Upon contact with a fluid such as a biological fluid, buffer, or aqueous solvent, the excipient may dissolve and/or absorb into the non-fouling layer. Accordingly, when exposed to an aqueous fluid such as, for example, a biological sample, the detection agent may be solubilized and released into the sample and may bind to a pathogen present in a pathogen region. The excipient may also further stabilize the detection agent during storage.

The detection agent can be a peptide, a protein, a carbohydrate, a lipid, a small molecule ligand, or a combination thereof. In some embodiments, the detection agent includes a peptide, a protein, or a combination thereof. In some embodiments, the detection agent includes a peptide or a protein. In some embodiments, the detection agent includes an extracellular receptor protein.

The detection agent is capable of specifically binding at least one pathogen. In some embodiments, the detection agent is capable of binding each, individual pathogen. Having a detection agent capable of binding each, individual pathogen can decrease the complexity and improve the efficacy of the disclosed devices and methods thereof. The detection agent is generally one member of a pathological binding pair, where a pathogen is the other member. For example, the detection agent can be an extracellular receptor that binds and/or facilitates entry of a pathogen into a host or host cell. In some embodiments, the detection agent includes an extracellular receptor that is a pathological binding partner of at least one pathogen. In some embodiments, the detection agent incudes an extracellular receptor that is a pathological binding partner of each pathogen. Example extracellular receptors include, but are not limited to, angiotensin-converting enzyme 2 (ACE2), adeno-associated virus receptor (AAVR), heparan sulfate, and phosphatidylserine (PS) receptors. In some embodiments, the detection agent includes ACE2, AAVR, heparan sulfate, PS receptors, or a combination thereof. In some embodiments, the detection agent includes ACE2 or a variant thereof.

The detection agent can further include a detectable moiety that, directly or indirectly, provides a detectable signal. Example detection moieties include, but are not limited to, fluorophores, chromophores, radiolabel s, polynucleotides, small molecules, enzymes, nanoparticles, and upconverters. In some embodiments, the detection moiety may be a fluorophore such as a cyanine (e.g., CyDyes such as Cy3 or Cy5), a fluorescein, a rhodamine, a coumarin, a fluorescent protein or functional fragment thereof, or it may include a small molecule such as biotin, or it may include gold, silver, or latex particles. In some embodiments, the detection agent includes a detection moiety selected from the group consisting of a chromophore, a fluorophore, a radiolabel, a polynucleotide, a small molecule, an enzyme, a nanoparticle, a microparticle, a quantum dot, and an upconverter.

The excipient is a molecule or a combination of molecules that is selected as to allow for a stable, but non-permanent, association between the detection agent and the non-fouling layer. In some embodiments, the excipient can be partially soluble, substantially soluble or soluble in an aqueous solution (e.g., buffer, water, sample, biological fluid, etc.). In such embodiments, the excipient can be selected from the non-limiting examples of salts, carbohydrates (e.g., sugars, such as glucose, fucose, fructose, maltose and trehalose), polyols (e.g., mannitol, glycerol, ethylene glycol), emulsifiers, water-soluble polymers, and any combination thereof. Such excipients are well known in the art and can be selected based on the interaction between the excipient and detection agent, the excipient and the brush-like polymer, the solubility of the excipient in a particular medium, and any combination of such factors.

In some embodiments, the excipient includes a salt, a carbohydrate, a polyol, an emulsifier, a water soluble polymer, or a combination thereof. In some embodiments, the excipient includes a salt, a carbohydrate, a water soluble polymer, or a combination thereof. In some embodiments, the excipient includes a salt, a carbohydrate, or a combination thereof. In some embodiments the excipient includes PEG. In some embodiments, the excipient includes trehalose.

The detection agent and excipient may be printed onto the non-fouling layer to form a detection region. The detection region(s) can be arranged in any particular manner and can include any desirable shape or pattern such as, for example, spots (e.g., of any general geometric shape), lines, or other suitable patterns that allow for identification of the detection region on the surface of the polymer and substrate. In some embodiments, a plurality of detection regions can be arranged in a predetermined pattern such that the identity of the detection agent is associated with a specific location on the non-fouling layer. The detection regions may be formatted in a manner to ensure that the detection regions are dissolved upon contact with a biological sample. In some embodiments, twelve separate detection regions are printed as spots, where the twelve detection regions surround the pathogen regions.

The detection regions can be arranged as an array on the non-fouling layer. When an array is formed by the deposition of multiple detection regions at discrete locations on the non-fouling layer, detection agent densities of 1, 3, 5, 10, 100 or up to 1000 detection agents locations per cm² can be made. Modern non-contact arrayers can be used in the deposition step to produce arrays having up to 1,000,000 detection agent locations per cm². For example, using dip-pen nanolithography, arrays with up to 1 billion discrete detection agent locations per cm² can be prepared. In some embodiments, the detection agent is present on the non-fouling layer at about 1 detection agent/cm² to about 1,000,000,000 detection agent/cm², such as about 1 detection agent/cm²to about 1,000,000 detection agents/cm², about 1 detection agent/cm²to about 500,000 detection agents/cm², about 10 detection agents/cm² to about 800,000 detection agents/cm², about 10 detection agents/cm² to about 100,000 detection agents/cm², about 10 detection agents/cm² to about 50,000 detection agents/cm², about 1 detection agent/cm² to about 10,000 detection agents/cm², or about 1 detection agent/cm² to about 1,000 detection agents/cm². It will be appreciated that the specific molecular species at each detection region can be different, or some can be the same (e.g., to provide some redundancy or control), depending upon the particular application, as described herein. In some embodiments, each detection region includes the same detection agent.

In some embodiments, for example when the biological fluid is a blood sample, the detection region may include an anticoagulant to prevent the blood from clotting. Example anticoagulants include, but are not limited to, vitamin K antagonists such as coumadin, heparins, and low molecular weight heparins.

### E. Other Elements

In some embodiments, the device may further include an agent to demarcate a patterned region on the non-fouling layer, such that a fluid (e.g., a biological sample) can remain confined to a specified region on the non-fouling layer such that it contacts the pathogen region and the detection region. Such an agent may be, for example, a hydrophobic ink printed on the non-fouling layer prior to the deposition of the pathogen and the components of the detection region. Alternatively, the agent may be a wax. In other embodiments, the sample may be contained or directed on the device through selection of an appropriate geometry and/or architecture for the substrate, for example, a geometry that allows the sample to diffuse to the regions including the pathogen and the components of the detection region. In some embodiments the substrate may include a well, or a series of interconnected wells.

In some embodiments, the device may further include regions printed with control agents. For example, the pathogen regions can include control regions printed alongside the pathogen regions to verify the activity of the detection agent and to normalize the signal from the detection moiety, such as fluorescence intensities.

The disclosed device can also be adapted to a microfluidics-based device. Further description of the device being adapted to a microfluidics-based device and the resultant structures are disclosed in International Patent Application No. PCT/US2021/046833 (published as WO2022/040495), which is incorporated herein in its entirety by reference.

### 3. METHODS

### A. Detecting a Neutralizing Antibody

Also disclosed herein are methods of detecting neutralizing antibodies using the disclosed devices. The method can include contacting a biological sample with a device. Example biological samples include, but are not limited to, blood, serum, plasma, and saliva. In some embodiments, the biological sample is blood, serum, or plasma. In some embodiments, the biological sample is blood or saliva. The sample can be diluted in a buffer prior to contacting the device. However, in some embodiments, the sample is undiluted and added directly to the device. In addition, the volume of the biological sample can be about 30 μL to about 1 mL, such as about 30 μL to about 900 about 50 μL to about 1 mL, about 40 μL to about 600 about 30 μL to about 500 μL, or about 50 μL to about 700 μL. In some embodiments, the volume of the biological sample is greater than 30 μL greater than 40 μL greater than 50 μL, or greater than 100 μL. In some embodiments, the volume of the biological sample is less than 1 mL, less than 900 μL, less than 800 μL, or less than 700 μL.

The biological sample can come from a subject. The subject can have a varying status with respect to exposure to a pathogen and being vaccinated against the pathogen. For example, the subject may have been vaccinated against the pathogen, the subject may have been exposed to the pathogen without being vaccinated against the pathogen, or the subject may have been exposed to the pathogen and vaccinated against the pathogen. Depending on the status of the subject, the subject may have neutralizing antibodies induced by the vaccine against the pathogen, have neutralizing antibodies induced by the native immune system (e.g., without the aid of a vaccine), or both. The disclosed methods can be used to detect neutralizing antibodies in a biological sample from the subject in these scenarios where the biological sample may or may not have a neutralizing antibody present. In addition, the biological sample may have more than one neutralizing antibody present. Accordingly, the disclosed methods can be used to assess a vaccine's effectiveness and/or a subject's native immune response, e.g., for inducing neutralizing antibodies, against a pathogen as well as its variants. In some embodiments, the subject is human.

The method can further include detecting the presence of a neutralizing antibody in the biological sample against a plurality of pathogens. Advantageously, the method can detect the presence of a neutralizing antibody in the biological sample against a plurality of pathogens simultaneously. For example, the method can detect the presence of a neutralizing antibody in the biological sample against each, individual pathogen at the same time, such as within 1 second, 5 seconds, 10 seconds, 30 seconds, 1 minute, or 5 minutes from when a neutralizing antibody is detected against the first pathogen being assessed to when a neutralizing antibody is detected against the last pathogen being assessed.

The method can be used to detect a neutralizing antibody in a significantly faster time compared to presently used assays. For example, detecting the presence of a neutralizing antibody can occur in about 10 minutes to about 1 hour after the biological sample contacts the device, such as about 15 minutes to about 1 hour after the biological sample contacts the device, about 20 minutes to 1 hour after the biological sample contacts the device, about 15 minutes to about 50 minutes after the biological sample contacts the device, about 20 minutes to about 40 minutes after the biological sample contacts the device, or about 10 minutes to about 30 minutes after the biological sample contacts the device. In some embodiments, detecting the presence of a neutralizing antibody occurs in less than or equal to 1 hour, less than or equal to 55 minutes, less than or equal to 50 minutes, less than or equal to 45 minutes, less than or equal to 40 minutes, less than or equal to 35 minutes, or less than or equal to 30 minutes after the biological sample contacts the device. In some embodiments, detecting the presence of a neutralizing antibody occurs in greater than or equal to 10 minutes, greater than or equal to 15 minutes, greater than or equal to 20 minutes, or greater than or equal to 25 minutes after the biological sample contacts the device.

The detected neutralizing antibody can be an antibody in the subject that was induced by a vaccine, induced by the native immune system (e.g., sans vaccine), or both. Accordingly, the neutralizing antibody can be one that is neutralizing of a pathogen, and more particularly, one that is neutralizing of a pathogen on the device. As such, the neutralizing antibody can be an anti-pathogen antibody, an anti-viral antibody, and the like. In some embodiments, the neutralizing antibody includes an anti-SARS-CoV-2 antibody. In some embodiments, the neutralizing antibody is an anti-SARS-CoV-2 antibody.

Following exposure of a device described herein to a biological sample (e.g., a biological fluid), a signal from the detection agent may be detected using any suitable method known in the art. Example methods include, but are not limited to, visual detection, fluorescence detection (e.g., fluorescence microscopy), scintillation counting, surface plasmon resonance, ellipsometry, atomic force microscopy, surface acoustic wave device detection, autoradiography, and chemiluminescence. As one of skill in the art will appreciate, the choice of detection method will depend on the specific detection agent employed. In some embodiments, the detection method is fluorescence. Prior to detection, the method may include a washing step. For example, the device may be washed with a buffer, such as one including a surfactant (e.g., Tween) and phosphate buffered saline.

If a neutralizing antibody is detected in the biological sample, it can be further stratified into different groups based on its neutralizing ability. For example, the neutralizing antibody can be described qualitatively, such as protective or partially protective. The neutralizing antibody can also be described quantitatively, such as by % neutralizing compared to a control.

### B. Determining Neutralizing Activity of a Subject

In another aspect, disclosed herein are methods of determining neutralizing activity (e.g., having neutralizing antibody(s)) of a subject. As mentioned above, the subject can be a vaccinated subject, a non-vaccinated subject that has been exposed to a pathogen, or a subject that has been vaccinated and exposed to a pathogen.

The method can be one of determining the neutralizing activity of a vaccine. The method can include obtaining a biological sample from a subject that has received a vaccine and contacting the biological sample with a device as disclosed herein. The method can further include detecting the presence of a neutralizing antibody induced by the vaccine for each pathogen, wherein the presence of the neutralizing antibody is detected by inhibiting the binding of the detection agent to each pathogen.

The vaccine can be any suitable vaccine against a pathogen. In some embodiments, the vaccine includes a vaccine against a pathogen that engages with an extracellular receptor binding partner. In some embodiments, the vaccine includes a vaccine against a viral pathogen or bacterium pathogen. In some embodiments, the vaccine includes a vaccine against a viral pathogen. In some embodiments, the vaccine includes a vaccine against SARS-CoV-2. The vaccine can also be produced or provided by a number of different pharmaceutical companies, including but not limited to, Pfizer, Moderna, and Johnson & Johnson. In addition, the subject can have received the vaccine for a varying amount of time prior to testing on the device. For example, the subject may have had the vaccine days, weeks, or months prior to testing on the device.

The method can also be one of determining the neutralizing activity of a subject exposed to a pathogen but not having received a vaccine against the pathogen. The method can include obtaining a biological sample from a subject that has been exposed to a pathogen and contacting the biological sample with a device as disclosed herein. The method can further include detecting the presence of a neutralizing antibody induced by the subject's immune system in response to the pathogen for each pathogen on the device, wherein the presence of the neutralizing antibody is detected by inhibiting the binding of the detection agent to each pathogen.

Methods of determining neutralizing activity of a subject are useful for screening a subject against a viral pathogen and a plurality of its variants. Accordingly, one can determine the neutralizing effectiveness of a vaccine or exposure to the viral pathogen itself in protecting the subject from the viral pathogen and variants thereof. In some embodiments, each pathogen (on the device) corresponds to an individual virus or a variant thereof. This latter aspect regarding screening against an individual virus and its variants can also be applied to the above methods of detecting a neutralizing antibody.

The methods disclosed herein can aid in tailoring a treatment plan for the subject. Accordingly, the method can further include a treatment step. For example, the methods can aid the subject in determining what type of treatment (e.g., a mAb treatment, a specific vaccine, etc.) the subject needs against a pathogen based on the results of the assay.

As methods of determining neutralizing activity of a subject also include methods of detecting neutralizing antibodies, the description of the methods of detecting neutralizing antibodies can be applied to the methods of determining neutralizing activity of a subject. In addition, the description of methods of determining neutralizing activity of a subject may also be applied to methods of detecting neutralizing antibodies. Furthermore, the description of the device, substrate, non-fouling layer, pathogen region, detection region, and other elements of the device above may be applied to the disclosed methods.

### 4. KITS

Also disclosed herein are kits that can be used for, e.g., detecting a neutralizing antibody in a biological sample. The kit can include a disclosed device, at least one buffer, and one or more packages, receptacles, delivery devices, labels, or instructions. The kit may also include other reagents to facilitate using the device and methods thereof. The choice of buffers and reagents will depend on the particular application, e.g., setting of the assay (point-of-care, research, clinical), analyte(s) to be assayed, the detection moiety used, etc.

In addition, the kit may include a packaging configured to contain the device and the buffer. The packaging may be a sealed packaging, such as a sterile sealed packaging. By “sterile” it is meant that there are substantially no microbes (such as fungi, bacteria, viruses, spore forms, etc.). In some embodiments, the packaging may be configured to be sealed, e.g., a water vapor-resistant packaging, optionally under an air-tight and/or vacuum seal.

Following construction of the device, it can be optionally dried, e.g., by mild desiccation, blow drying, lyophilization, or exposure to ambient air at ambient temperature, for a time sufficient for the article to be dry or at least macroscopically dry. Once the device is dry or at least macroscopically dry, it may be sealed in a container (e.g., such as an impermeable or semipermeable polymeric container) in which it can be stored and shipped to a user. Once sealed in a container, the device may have, in some embodiments, a shelf life of at least 2 to 4 months, or up to 6 months or more, when stored at a temperature of 25° C. (e.g., without loss of more than 20%, 30% or 50% of binding activity).

The kits may further include instructions for using the device. These instructions may be present in the kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another form for the instructions could be a computer readable medium, e.g., computer-readable memory (e.g., flash memory), etc., on which the information has been recorded or stored. Yet another form for the instructions that may be present is a website address which may be used via the Internet to access the information at a removed site. Any convenient means may be present in the kits.

The disclosed invention has multiple aspects, illustrated by the following non-limiting examples.

### 5. EXAMPLES

### Example 1

### Materials & Methods

Study Design. First, monoclonal antibodies with known neutralizing activity were evaluated. For these monoclonal antibodies, the assay was benchmarked against a live virus microneutralization assay (Isolate USA-WA1/2020, NR-52281) to establish concordance between CoVariant-SCAN and the microneutralization assay using Pearson's r correlation (conducted in GraphPad Prism). Plasma was also examined from healthy controls, convalescent individuals with varying disease severity, and COVID-19 vaccine recipients. Sample sizes were chosen based on availability of clinical samples in existing repositories or through commercial vendors. To examine the difference between ACE2-RBD blocking among different groups or different variants, one-way ANOVA was performed with post-hoc testing (GraphPad Prism). A subset of convalescent samples had previously been characterized using the live virus neutralization assay, and the results from the CoVariant-SCAN were hence benchmarked using those samples. All experiments were performed in multiple replicates, as indicated throughout the materials and methods and figure legends. As this was an observational study, experiments were not randomized or blinded; however, all clinical samples were identically tested and analyzed.

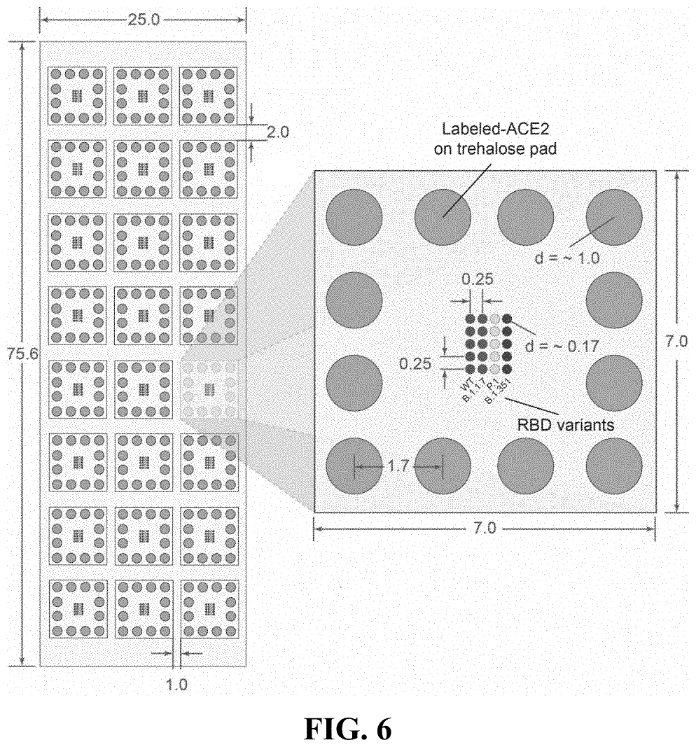

CoVariant-SCAN assay fabrication. Glass substrates were coated with a POEGMA polymer brush with a thickness of ˜50 nm by surface-initiated atom transfer radical polymerization (SI-ATRP). Next, recombinant SARS-CoV-2 RBD proteins for WT (Sino Biological, catalog #40592-V08H), B.1.1.7 (Sino Biological, catalog #40592-V08H82), P.1 (Sino Biological, catalog #40592-V08H86), and B.1.351 (Sino Biological, catalog #40592-V08H85) variants were immobilized on the POEGMA-coated glass slides using a Scienion S11 sciFLEXARRAYER (Scienion AG) inkjet printer. Columns of five ˜180 μm diameter capture spots for each RBD variant were printed at a concentration of 0.8 mg/mL (FIG. 6). Surrounding the capture spots, twelve 1 mm-diameter trehalose spots were printed using a BioDot AD1520 printer (BioDot Inc.) loaded with a 10% (w/v) trehalose solution (˜100 nL drop volume). Next, Alexa Fluor 647 labeled human ACE2 (Sino Biological, catalog #10108-H05H) were deposited on top of the excipient pads using the BioDot printer at a concentration of 0.02 mg/mL. Twenty-four assays with this configuration were printed on each 75.6×25.0×1.0 mm glass slide in a 3×8 array. CoVariant-SCAN assays were stored under vacuum for at least 24 h before use. For experiments with the B.1.617.2 variant, an additional column of 5 capture spots was inkjet printed onto the POEGMA surface (Sino Biological, catalog #40592-V08H90). This device structurally and mechanistically differs from the immunoassay described in D. Y. Joh et al., Inkjet-printed point-of-care immunoassay on a nanoscale polymer brush enables subpicomolar detection of analytes in blood. Proc Natl Acad Sci USA 114, E7054-E7062 (2017), which is incorporated herein in its entirety by reference.

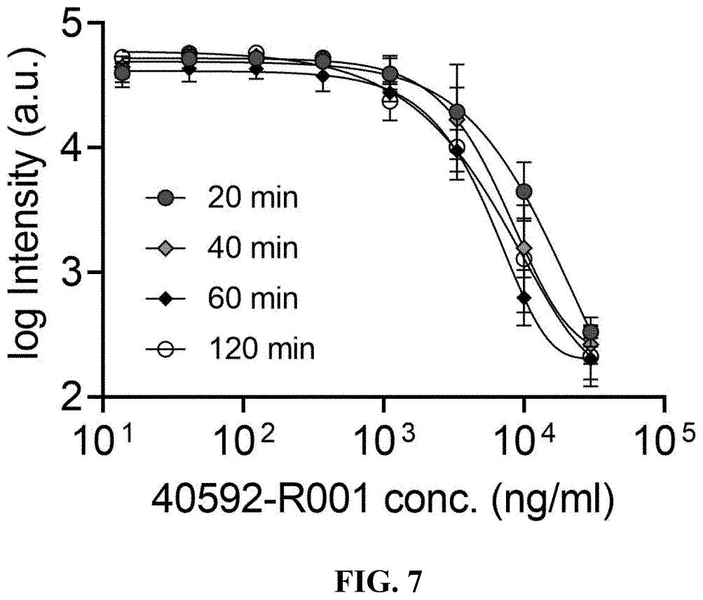

Analytical testing using the CoVariant-SCAN assay. CoVariant-SCAN chips were secured in a 96-well microarray hybridization cassette or adhered to a laser-cut acrylic that separates the chip into 24 separate wells. To perform the assay, 60 μL of sample were added directly to an assay well, covered, and incubated at room temperature for 1 h. Although 1 h was chosen, the incubation time does not drastically impact the results (FIG. 7) and assays could be incubated for 20 min or possibly shorter. After incubation, samples were aspirated and chips were rinsed in wash buffer (0.1% Tween-20 in 1× PBS), dried, and then scanned with an Axon Genepix 4400 tabletop scanner (Molecular Devices LLC). PHS that was collected pre-pandemic was tested on each chip to serve as a negative control. The average fluorescence intensity at each capture spot was quantified using the Genepix Pro 7 analysis software. All fluorescence intensities were log transformed prior to analysis. To calculate percentage blocked, the following formula was used:

$$\% blocking = 100 \times ( 1 - \frac{x - B}{NC - B} )$$

where x is the log-transformed intensity, B is a constant (2.301) representing the background fluorescence signal, and NC is the log-transformed intensity of the negative control samples, which was calculated separately for each experiment and for each variant.

All mAbs were diluted in PHS collected pre-pandemic. For experiments with recombinant and EUA-approved mAbs, each dose was run in triplicate. For experiments with the convalescent patient-derived mAbs, each dose was run in duplicate. A 7-point dose-response curve was tested for all mAbs with a starting concentration of 30 μg/mL. All data were plotted using GraphPad Prism version 9.1.1 (GraphPad Software). Regression analysis was performed in GraphPad using an asymmetric, five parameter logistic equation for dose-response experiments.

All individual donor plasma samples (pre-pandemic healthy controls, mild, ICU, vaccine recipients) assayed during this study were tested identically. Plasma samples were thawed from −80° C. storage and allowed to reach room temperature before testing on the CoVariant-SCAN. Each sample was tested undiluted in duplicate to assess the reproducibility of the assay, where a strong correlation between replicates was found. The percentage blocked was calculated as described above and plotted as the mean of duplicate assays.

Source of monoclonal neutralizing antibodies. Convalescent donor-derived monoclonal antibodies isolated as previously described, see D. R. Martinez et al., A broadly neutralizing antibody protects against SARS-CoV, pre-emergent bat CoVs, and SARS-CoV-2 variants in mice. bioRxiv, 2021.2004.2027.441655 (2021) and D. Li, et al., The functions of SARS-CoV-2 neutralizing and infection-enhancing antibodies in vitro and in mice and nonhuman primates. bioRxiv, 2020.2012.2031.424729 (2021)—both of which are incorporated herein by reference in their entirety, were acquired from the Duke Human Vaccine Institute Pandemic Prevention Program. The recombinant mAbs were purchased commercially (Sino Biological, catalog #40591-MM43, catalog #40592-MM57, catalog #40592-R0004, catalog #40592-R001; ACRO Biosystems, catalog #SAD-535; R&D Systems, catalog #MAB105802). Regeneron therapeutic mAbs were acquired from the Duke University Medical Center Pharmacy. All mAbs are summarized in Table 1.

TABLE 1
Monoclonal antibody summary

| Antibody ID | Source | Specificity | Species |
|---|---|---|---|
| DH1143 | Convalescent-patient derived | RBD | Human |
| DH1154 | Convalescent-patient derived | RBD | Human |
| DH1126 | Convalescent-patient derived | RBD | Human |
| DH1179 | Convalescent-patient derived | RBD | Human |
| DH1184 | Convalescent-patient derived | RBD | Human |
| DH1042 | Convalescent-patient derived | RBD | Human |
| DH1043 | Convalescent-patient derived | RBD | Human |
| DH1161.1 | Convalescent-patient derived | RBD | Human |
| DH186 | Convalescent-patient derived | RBD | Human |
| DH1041 | Convalescent-patient derived | RBD | Human |
| DH1047 | Convalescent-patient derived | RBD | Human |
| DH1109 | Convalescent-patient derived | RBD | Human |
| DH1191 | Convalescent-patient derived | RBD | Human |
| DH1139 | Convalescent-patient derived | RBD | Human |
| DH1169 | Convalescent-patient derived | RBD | Human |
| DH1172 | Convalescent-patient derived | RBD | Human |
| DH1096 | Convalescent-patient derived | RBD | Human |
| DH1127 | Convalescent-patient derived | RBD | Human |
| DH1152 | Convalescent-patient derived | RBD | Human |
| 40592-MM57 | Sino Biological Inc. | RBD | Mouse |
| SAD-S35 | Acro Biosystems | RBD | Human |
| 40592-R001 | Sino Biological Inc. | RBD | Rabbit |
| 40591-MM43 | Sino Biological Inc. | S1/RBD | Mouse |
| MAB105802 | R&D Systems Inc. | RBD | Mouse |
| 40592-R0004 | Sino Biological Inc. | RBD | Rabbit |
| REGN10933 | Regeneron Pharmaceuticals Inc. | RBD | Human |
| REGN10987 | Regeneron Pharmaceuticals Inc. | RBD | Human |

Clinical samples. De-identified plasma samples from severe COVID-19 cases requiring hospitalization in the ICU were accessed from the Duke COVID-19 ICU biorepository (Pro00101196) approved by the Duke Health Institutional Review Board (IRB). For the mild and moderate/hospitalized cohorts, plasma samples from patients with confirmed SARS-CoV-2 infection were identified through the Duke University Health System (DUHS) or the Durham Veterans Affairs Health System (DVAHS) and enrolled into the Molecular and Epidemiological Study of Suspected Infection (MESSI, Pro00100241). Samples were accessed via the same exempt protocol (Pro00105331). Clinical severity was measured according to the National Institutes of Health clinical grading scale. Patients with severe infection have SpO₂<94% on room air at sea level, a ratio of arterial partial pressure of oxygen to fraction of inspired oxygen (PaO₂/FiO₂)<300 mm Hg, respiratory frequency>30 breaths/min, or lung infiltrates>50%. The moderate/hospitalized disease cohort was defined as individuals who experienced symptoms requiring hospitalization but did not require admission to the ICU. For 4 individuals, symptom onset was unknown; however, samples were taken at least 3 weeks after hospitalization. For calculation of the mean days since symptom onset, those samples were excluded. Those with mild disease may have any of the various signs and symptoms of COVID-19 (e.g., fever, cough, sore throat, malaise, headache, muscle pain, nausea, vomiting, diarrhea, loss of taste and smell) but do not have shortness of breath, dyspnea, or abnormal chest imaging. Microneutralization assessments of clinical samples were run under Pro00105165. 28 pre-pandemic negative control plasma samples were purchased commercially (Lee BioSolutions Inc. and Innovative Research Inc.). Plasma samples from vaccine recipients were either collected under the MESSI protocol or purchased commercially (Innovative Research Inc and RayBiotech Life Inc). For the experiments with the B.1.617.2 variant, fresh blood from 19 individuals was collected in EDTA coated tubes under a Duke IRB protocol (Pro00106419). Blood was processed to plasma by centrifugation at 1800 rcf for 15 minutes at 4° C. Plasma was aspirated from the top layer, aliquoted, and stored at −80° C. prior to assaying on the CoVariant-SCAN. All samples were de-identified and tested under an exempt protocol (Pro00105331). Each cohort is summarized in Table 2.

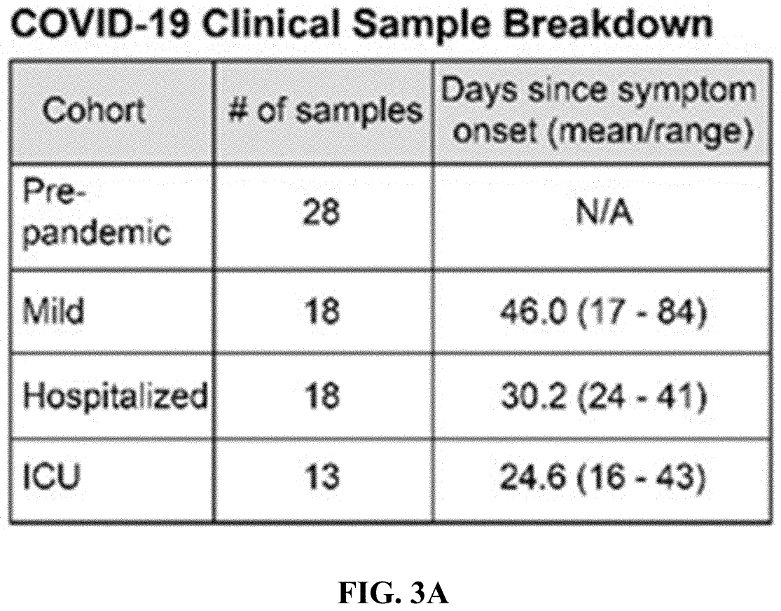

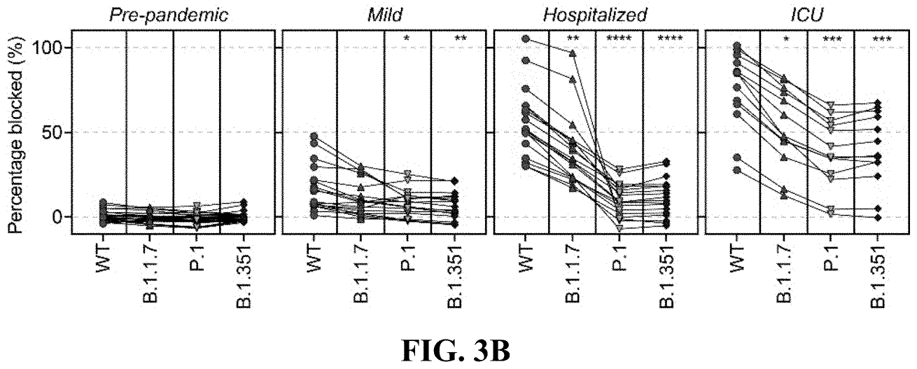

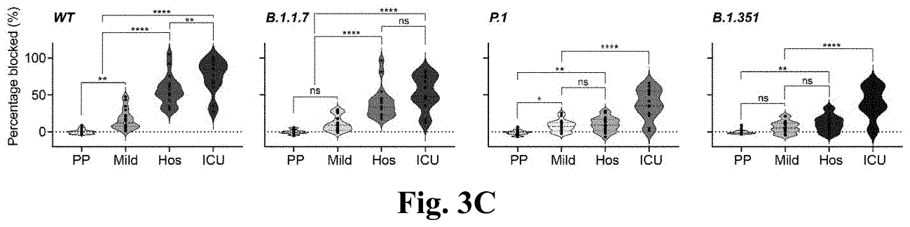

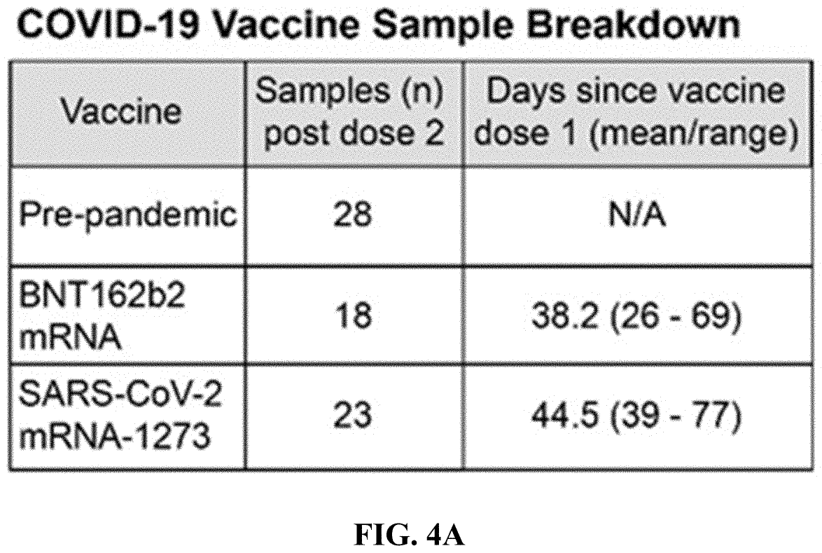

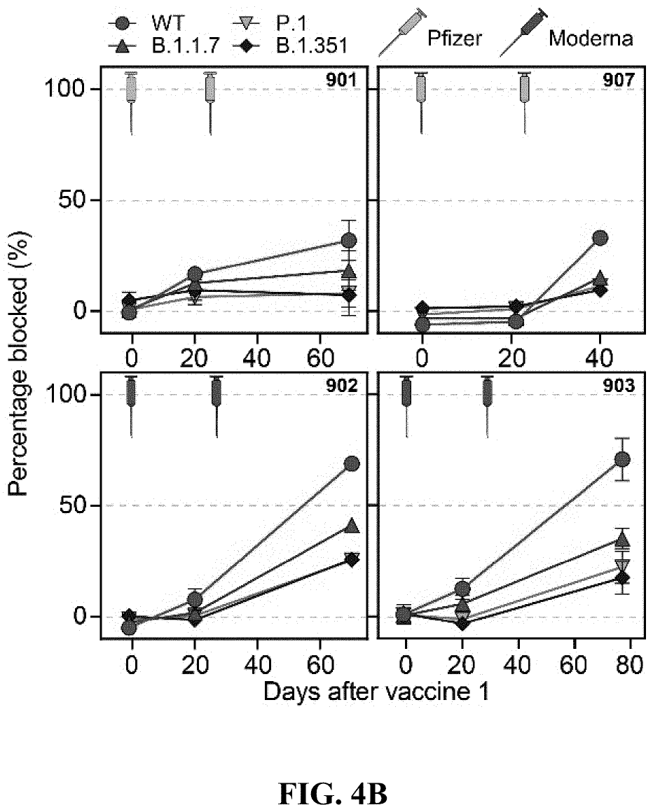

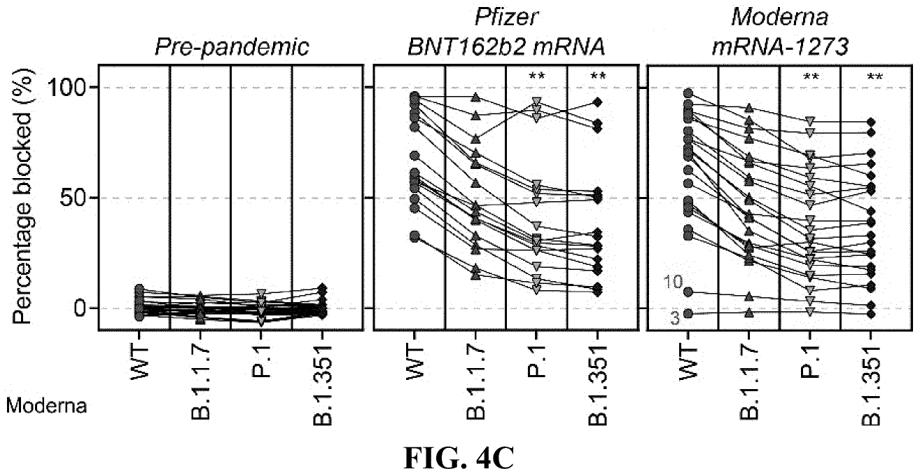

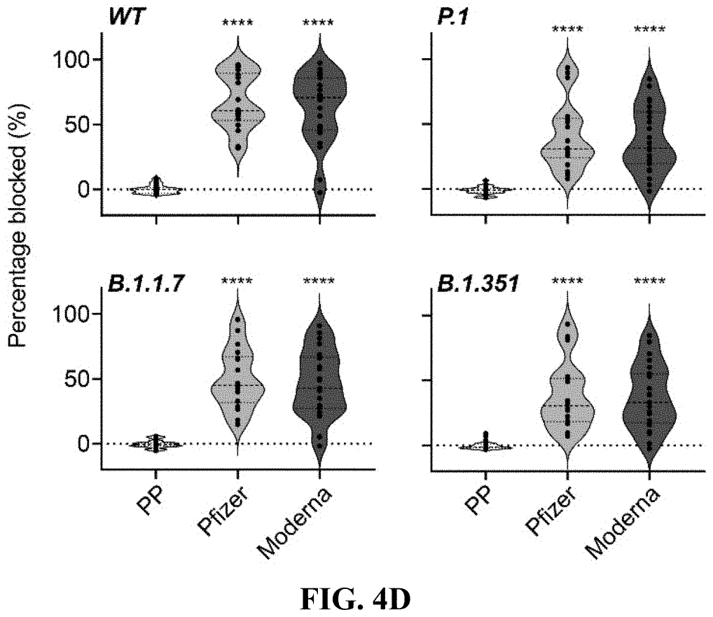

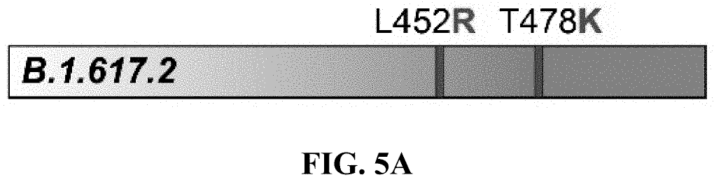

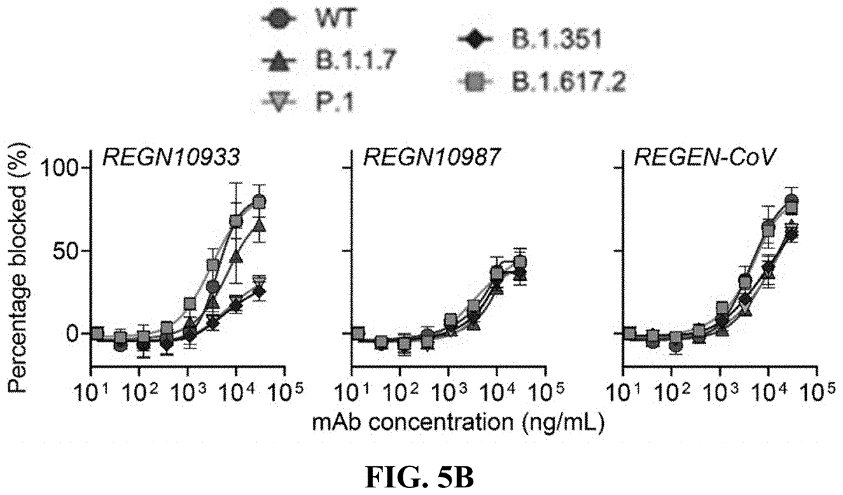

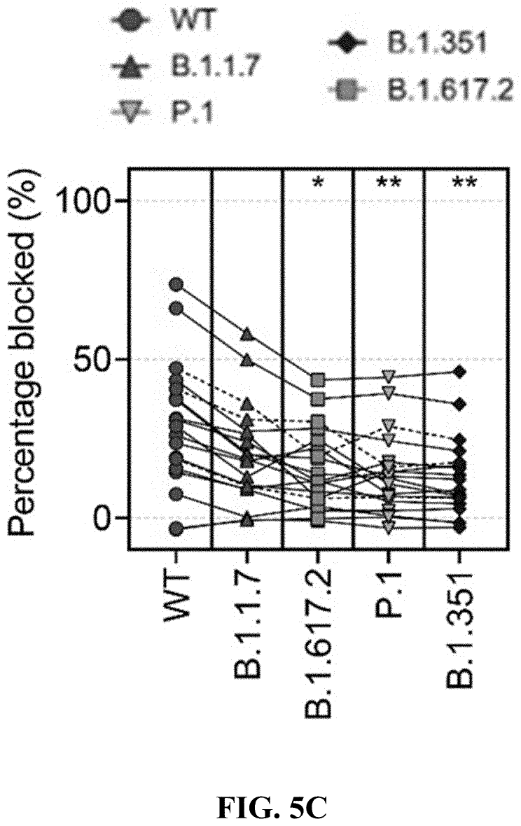

TABLE 2
Clinical sample summary

| | | | Gender | Days since | Days relative |
|---|---|---|---|---|---|
| | Number of | Age | breakdown | symptom onset | to vaccine 1 |
| Cohort | samples | (mean/range) | (M:F) | (mean/range) | (mean/range) |
| Pre-pandemic | 28 | 52.8 (17-73) | 18:10 | N/A | N/A |
| (FIG. 3B) | | | | | |
| Mild COVID-19 | 18 | 32.8 (20.1-61.4) | 10:8 | 46 (17-84) | N/A |
| (FIG. 3C) | | | | | |
| Moderate COVID-19 | 18 | 52.2 (32.1-71.3) | 7:11 | 30.2 (24-41) | N/A |
| (FIG. 3D) | | | | | |
| Severe COVID-19 | 13 | 54.1 (41-66) | 7:6 | 24.9 (17-43) | N/A |
| (FIG. 3E) | | | | | |
| Pfizer (FIG. 4D) | 18 | 45.7 (32-60.2) | 7:11 | N/A | 38.2 (26-69) |
| Moderna (FIG. 4E) | 23 | 49.9 (29-83.7) | 10:13 | N/A | 44.5 (39-77) |
| Johnson & Johnson | 3 | 43.7 (33.5-50.7) | 2:1 | N/A | 15.3 (15-16) |
| Longitudinal | 12 | 40.25 (32-48) | 9:3 | N/A | 27.8 (−1-77) |
| samples (FIG. 4B) | | | | | |
| Plasma samples | 19 | 32.1 (20-52) | 10:9 | N/A | 111.4 (91-126) |
| from freshly | | | | | |
| collected blood | | | | | |
| (FIG. 5C) | | | | | |

Indirect assay. To fabricate indirect assays to detect anti-RBD antibodies, SARS-CoV-2 WT RBD was inkjet printed onto POEGMA-coated glass slides, as described above. Chips were placed in a microarray cassette to separate the chip into 24 separate arrays. Next, 60 μL of each sample was added to arrays in duplicate and incubated for 45 min. After incubation, each assay was washed 3-times with 100 μL of wash buffer (0.1% Tween-20 in 1× PBS) and then 60 μL of Alexa Fluor 647 fluorescently labeled mouse anti-human IgG (Southern Biotech, catalog #9040-01) at 2 μg/mL was added to each array for 15 min. Finally, slides were washed, dried, and imaged on an Axon Genepix tabletop scanner. For experiments with non-neutralizing mAbs isolated from convalescent patients, each mAb was spiked into PHS diluted 1:10 (1% w/v bovine serum albumin, 0.05% Tween-20 in PBS diluent solution) at concentrations ranging from 4.1 ng/mL to 3 μg/mL. Dose-response curves were fit in GraphPad using an asymmetric, five parameter logistic equation. For experiments with plasma samples from vaccine recipients and individual donor pre-pandemic healthy controls, samples were diluted 1:10 (1% w/v bovine serum albumin, 0.05% Tween-20 in PBS diluent solution) and then assayed as described.

Microneutralization assay. The SARS-CoV-2 virus (Isolate USA-WA1/2020, NR-52281) was deposited by the Centers for Disease Control and Prevention and obtained through BEI Resources, NIAID, NIH. SARS-CoV-2 Micro-neutralization (MN) assays were adapted from a previous study, see J. D. Berry et al., Development and characterization of neutralizing monoclonal antibody to the SARS-coronavirus. J Virol Methods 120, 87-96 (2004), which is incorporated herein by reference in its entirety, as follows. Recombinant antibodies or plasma samples were diluted two-fold and incubated with 100 TCID50 virus for 1 h. These dilutions were transferred to a 96-well plate containing 2×10⁴ Vero E6 cells per well. Following a 96 h incubation, cells were fixed with 10% formalin, and cytopathic effect (CPE) was determined after staining with 0.1% crystal violet. Each batch of MN includes a known neutralizing control antibody (Sino Biological, catalog #40150-D001). Data are reported as IC₅₀ or the inverse of the last concentration at which a test plasma protects Vero E6 cells.

Statistical analysis. All statistical analysis was performed using GraphPad Prism version 9.1.1 (GraphPad Software, Inc). All data were log-transformed for analysis. Regression analysis was performed in GraphPad using an asymmetric, five parameter logistic equation for dose-response experiments. One-way ANOVA was performed to establish statistical significance between different groups followed by post-hoc multiple comparison tests (Tukey or Dunnett's). Pearson r correlation was calculated to assess the degree of agreement between different assays and replicates.

### Example 2

### Neutralization by Monoclonal Antibodies

The potency of 28 mAbs against SARS-CoV-2 variants was assessed using the CoVariant-SCAN (Table 1). These included 20 mAbs derived from a convalescent individual, 6 recombinant mAbs, and 2 mAbs with Emergency Use Authorization (EUA): REGN10987 (imdevimab) and REGN10933 (casirivimab). The convalescent donor-derived mAbs were isolated from plasmablasts or reactive memory B cells from a SARS-CoV-2 infected individual 11-, 15- and 36-days post symptom onset. Recombinant mAbs—purchased commercially—were isolated from immunized mice (n=3) or rabbits (n=2), or from a SARS-CoV-2 infected patient (n=1). Finally, the Regeneron mAbs were isolated from humanized mice and recovered patients. All mAbs assayed on CoVariant-SCAN are specific to RBD. Each mAb was spiked into pooled human serum (PHS) collected prior to the COVID-19 outbreak at a starting concentration of 30 μg ml⁻¹ and a dilution series spanning three logs was evaluated on CoVariant-SCAN chips. In parallel, the 20 convalescent patient-derived mAbs were characterized using a live virus microneutralization assay to benchmark the CoVariant-SCAN assay. For the CoVariant-SCAN assay, the potency of each mAb was defined by two metrics: (1) the percentage of ACE2/RBD binding blocked at the highest concentration that was assayed (30 μg ml⁻¹); and (2) the mAb concentration that blocks at least 20% of ACE2 binding to the target RBD. The first definition was chosen to mimic how CoVariant-SCAN could be used at the point-of-care to assay undiluted samples. Conversely, the second definition more closely resembles a traditional inhibitory concentration measurement which requires testing a sample at multiple dilutions.

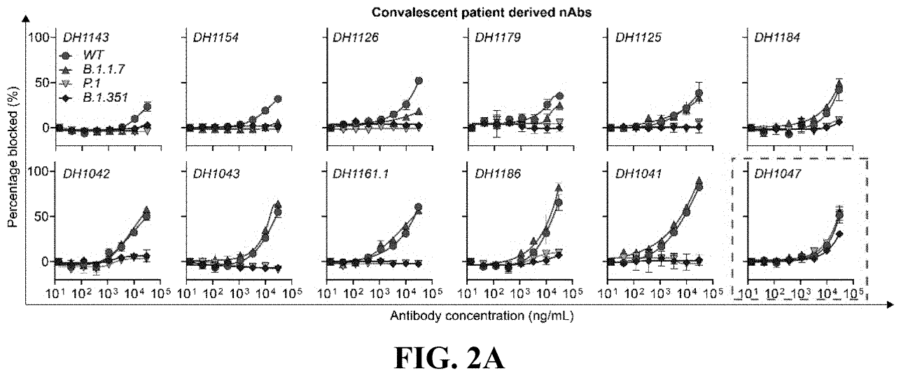

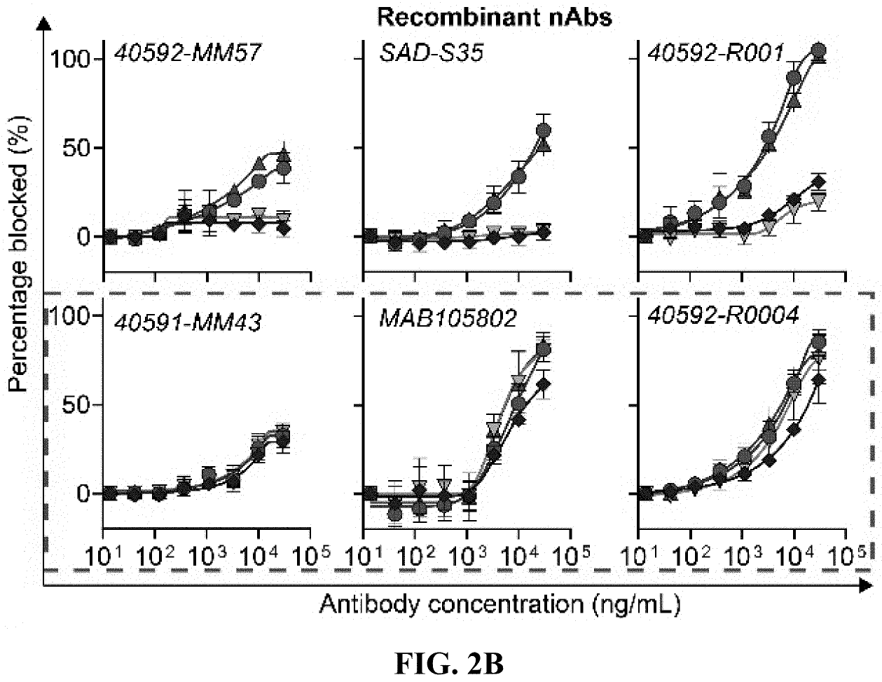

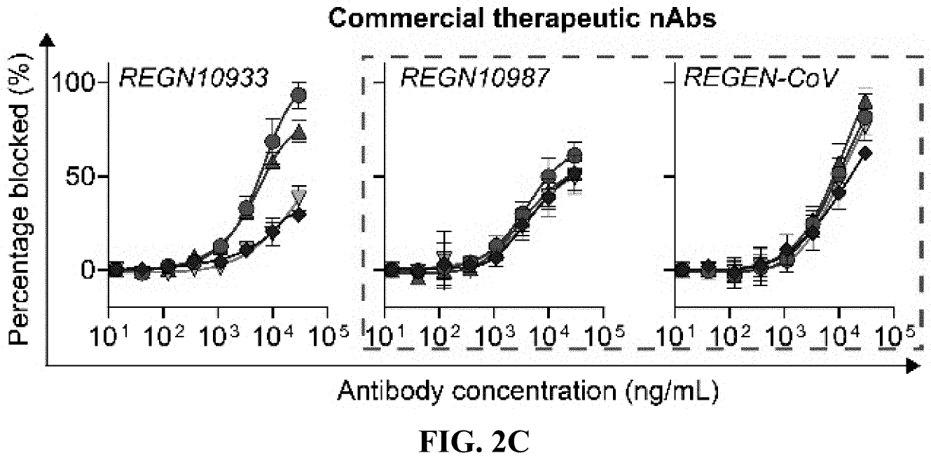

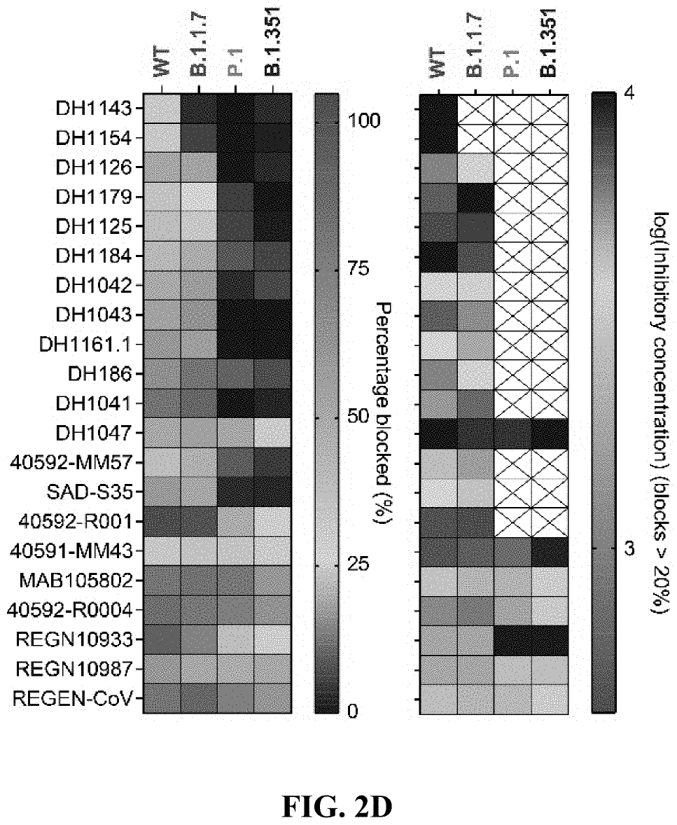

It was found that 12 out of 20 mAbs derived from convalescent individuals had neutralizing activity in the WT live virus assay. These 12 antibodies also blocked ACE2 binding to WT RBD in a dose-dependent manner in the CoVariant-SCAN assay (FIG. 2A). The 8 mAbs that were non-neutralizing in the live virus assay demonstrated weak or no blocking activity in CoVariant-SCAN despite having binding specificity to RBD, suggesting that the assay is specific to nAbs. In addition, all 20 convalescent-patient derived mAbs showed similar dose-response behavior when tested by an indirect assay, suggesting that their binding epitope—rather than their affinity for the RBD—is responsible for the differences in neutralizing/blocking activity. Good concordance was found between the potency measured on the CoVariant-SCAN, compared to the WT live virus 50% inhibitory concentration (IC₅₀), indicating that the test can reliably assess nAb activity. In addition, all 6 recombinant mAbs (FIG. 2B) and both EUA approved mAbs (FIG. 2C) demonstrated dose-dependent blocking of ACE2 binding to WT RBD.

In general, the mAbs assayed fell into one of two categories: (1) those that neutralize WT and B.1.1.7 similarly and have low or negligible potency towards P.1. and B.1.351, and (2) those with similar potency across all variants. The first category includes mAbs that likely target the ACE2-binding site within RBD, termed the receptor-binding motif (RBM). Numerous studies have demonstrated that mutations that occur within the RBM drastically impact neutralization for antibodies targeting the RBM. Of particular concern are mutations at residue E484—as is the case for B.1.351 and P.1—which have a large effect on plasma antibody binding and neutralization. Notably, REGN10933 which binds to the RBM has been shown to have diminished neutralization towards B.1.351 and P.1 variants, which is consistent with the results from CoVariant-SCAN. The second category contains mAbs that effectively blocked ACE2 binding to all variants similarly (enclosed in red dashed outlines). These mAbs likely target the “inner side” or the “outer side” of the RBD, as nAbs targeting these regions have been shown to retain neutralization activities against B.1.351 and P.1 variants. Of note, REGN10987, which targets the side of RBD has been shown to retain its neutralization activity towards B.1.351 and P.1. variants and was able to block ACE2 binding to each RBD variant in CoVariant-SCAN. When both EUA-approved mAbs were tested as a cocktail (REGEN-CoV), WT, B.1.1.7, P.1 and B.1.351 variants were all neutralized similarly and effectively. The potency of each mAb towards all variants is summarized in FIG. 2D. Collectively, these results suggest that CoVariant-SCAN can be used to screen potential mAb therapeutics against SARS-CoV-2 variants and can identify mAbs with broad potency or that act synergistically in mAb cocktails. Notably, an interesting finding that emerges from a comparison of the Regeneron antibody cocktail with the other 26 mAbs shows that compared to the rest of the mAbs tested in this study, the Regeneron cocktail offers robust protection against all three variants that is comparable to the protection conferred against WT SARS-CoV-2—against which these antibody drugs were originally developed.

### Example 3

### Neutralization by SARS-CoV-2 Infected Individuals

Plasma from COVID-19 positive patients was next assayed by CoVariant-SCAN. Plasma was obtained from 13 patients with severe presentation who required admission to an intensive care unit (ICU), 18 patients with moderate presentation who required hospitalization but not admission to an ICU, 18 patients with mild presentation who did not require hospitalization or have worsening symptoms, and 28 pre-pandemic healthy negative controls. Individuals in the mild cohort exhibited one or more of the following symptoms of COVID-19 (e.g., fever, cough, sore throat, malaise, headache, muscle pain, nausea, vomiting, diarrhea, loss of taste and smell), but did not experience shortness of breath, dyspnea, or abnormal chest imaging. All positive samples were collected at least 2 weeks after symptom onset once seroconversion is expected to have occurred, with a mean of 46.0 days for the mild cohort, 30.2 days for the hospitalized/moderate cohort, and 24.9 days for the ICU cohort (FIG. 3A). All COVID-19 positive samples were collected before SARS-CoV-2 variants were widely circulating in the US. For all samples, the percentage of ACE2-RBD blocked from undiluted samples was used as a proxy for antibody neutralization.

All 28 pre-pandemic negative controls showed negligible ACE2 blocking against each RBD variant (FIG. 3B). In the mild cohort, several patients were identified who developed nAbs against WT (FIG. 3C). There was a statistically significant difference in ACE2 blocking between all variants, as quantified by CoVariant-SCAN and determined by one-way ANOVA (F(3.68)=3.75, p=0.0149). Multiple comparisons by Dunnett's test revealed that the WT group exhibited a statistically significant higher percent blocking compared to both P.1 (p=0.0283) and B.1.351 (p=0.0078) groups, indicating that neutralization against P.1 and B.1.351 was diminished relative to WT. Conversely, there was no significant decrease in neutralization against B.1.1.7 (p=0.2068), indicating that B.1.1.7 can be cross neutralized by convalescent plasma with only a modest decrease in potency. The mean fold decrease in percent ACE2 binding relative to WT was 1.5-fold for B.1.1.7, 2.2-fold for P.1, and 2.7-fold for B.1.351 for the mild illness cohort. For the hospitalized cohort, there was a statistically significant difference between each variant on CoVariant-SCAN (FIG. 3D), as determined by one-way ANOVA (F(3.68)=33.41, p<0.0001). The percent blocking against WT was significantly higher compared to B.1.1.7 (p=0.0082), P.1 (p<0.0001), and B.1.351 (p<0.0001), with a mean fold decrease (relative to WT) of 1.4-fold, 5.6-fold, and 4.9-fold, respectively. For the ICU cohort, there was a statistically significant difference in ACE2 blocking between each variant (FIG. 3E), as determined by one-way ANOVA (F(3.52)=14.7, p<0.0001). Similar to the hospitalized cohort, blocking in the WT group was significantly higher than B.1.1.7 (p=0.0383), P.1 (p=0.0002) and B.1.351 (p=0.0005). The mean fold decrease in percent ACE2 binding relative to WT was 1.4-fold for B.1.1.7, 2.0-fold for P.1, and 1.9-fold for B.1.351 in the ICU cohort.

Next, the nAb blocking for each variant was compared across the four sample cohorts (FIG. 3F). For all variants, there was statistically significant higher blocking for ICU samples compared to pre-pandemic controls (p<0.0001) and the mild cohort (p<0.0001). This is consistent with observations from other studies that severely ill patients generate higher titers of nAb compared to those with a mild infection. Hospitalized patients developed higher levels of nAbs against all variants compared to pre-pandemic controls; however, there was no significant difference between hospitalized and mild cases for P.1 and B.1.351 variants. A similar trend can be seen in the mild cohort as compared to the ICU and hospitalized cohorts, though the overall magnitude of the humoral response is blunted. Patients in the mild cohort had significantly higher nAb blocking against WT compared to pre-pandemic controls (p=0.0028). For all other variants—B.1.1.7, P.1, and B.1.351—although some patients developed sufficient nAb levels to block ACE2 binding to all variants that were well above the baseline, only P.1 was statistically different when comparing mild infection versus pre-pandemic samples (p=0.03).

Collectively, the results largely confirm the findings of studies that used live virus or pseudovirus neutralization assays that convalescent plasma neutralizes B.1.1.7 similar to WT with only a modest decrease in potency, while activity against P.1 and B.1.351 is more severely diminished. In addition, individuals who developed more severe COVID-19 produced more nAbs with some degree of cross neutralization against all VOCs tested. To directly test the concordance between CoVariant-SCAN and a microneutralization assay, a separate set of ICU samples that had been previously characterized by a live virus microneutralization assay were measured by CoVariant-SCAN. A strong correlation was found between the results of CoVariant-SCAN and the microneutralization assay, confirming the validity of the assay.

### Example 4

### Neutralization by Vaccinated Individuals

Next, plasma was assayed from COVID-19 vaccine recipients with CoVariant-SCAN. Plasma was tested from 41 individuals, including individuals from whom longitudinal plasma samples were available (before first dose, after first dose, and after second dose). Of the 41 individuals, 18 received the BNT162b2 mRNA vaccine from Pfizer and 23 received the mRNA-1273 vaccine from Moderna. The average days since receiving vaccine dose one was 38.2 for Pfizer and 44.5 for Moderna. Since dose two, it was 18.1 and 28.0 days, respectively (FIG. 4A). Similar to the COVID-19 positive samples, all plasma samples were tested without processing or dilution, so the percentage of ACE2 blocked was used as a proxy for antibody neutralization.

Longitudinal samples were tracked from two individuals who received the Pfizer vaccine and two who received the Moderna vaccine at three time points during the immunization process: (1) pre-vaccine, (2) >2 weeks after dose one, and (3) >2 weeks after dose two (FIG. 4B). It was found that the CoVariant-SCAN could effectively track seroconversion in all four individuals and that all individuals developed nAbs that could block ACE2 binding to WT RBD, relative to their pre-vaccination control plasma sample. Antibody neutralization against B.1.1.7, P.1 and B.1.351 variants was attenuated relative to WT for all individuals, although it was still elevated compared to the pre-vaccine plasma samples. Notably, one dose of either Pfizer or Moderna vaccines did not yield sufficient nAb titers to block ACE2 binding against several variants, thus supporting the use of two-dose regimens to maximize neutralizing activity against WT and other VOCs.

Next, neutralization was examined against WT, and the B.1.1.7, P.1 and B.1.351 variants in individuals sampled at least one week after their second dose. Pre-pandemic negative control samples (from FIG. 3B) are also shown in FIG. 4C as a baseline reference. It was found that there was a statistically significant difference in neutralization against each variant in individuals receiving the Pfizer vaccine (FIG. 4D), as determined by one-way ANOVA (F(3.68)=5.14, p=0.0029). ACE2 blocking by nAbs was significantly higher for WT compared to P.1 (p=0.005) and B.1.351 (p=0.002) variants, while there was no significant difference compared to B.1.1.7 (p=0.0897). Similarly, in the Moderna cohort, neutralization was different across each variant (FIG. 4E), as determined by one-way ANOVA (F(3.88)=5.02, p=0.0029). ACE2 blocking by nAbs was lower for P.1 (p=0.004) and B.1.351 (p=0.003) variants relative to WT, and there was no statistically significant difference compared to B.1.1.7 (p=0.090). No significant difference was found in ACE2 blocking between the Pfizer and Moderna vaccines for any specific variant (FIG. 4F) and both vaccines significantly neutralized all variants tested relative to pre-pandemic negative control samples (adjusted p<0.0005). Plasma was also tested from 3 individuals who received the single-dose AD26.CoV2.S vaccine (co-developed by Johnson & Johnson/Janssen).

Interestingly, there was a relatively high amount of heterogeneity in the nAb response across all individuals tested. For example, some individuals developed nAbs that could block greater than 80% of ACE2 binding against all variants, while others (samples 3 and 10 in FIG. 4E) showed little to no neutralizing activity against any tested variant, despite developing anti-RBD antibodies when tested on an indirect assay that measures all IgG antibodies that bind to RBD. It is worth noting that the individuals with low nAb levels as measured by CoVariant-SCAN may still be protected from COVID-19 via mechanisms not directly related to ACE2-RBD blocking, which would require further investigation. Overall, the results from CoVariant-SCAN are consistent with other studies in that neutralization from vaccinee plasma against B.1.1.7 is essentially unchanged, while there is a significant loss in nAb activity against P.1 and B.1.351 variants, likely due to the E484K mutation. These findings may help explain COVID-19 breakthrough cases that have occurred due to infection with emerging variants even after immunization and support the continued development of variant-specific boosters.

The data shows that there is a considerable individual heterogeneity in nAb levels and that some individuals develop robust responses against all variants tested, while others may benefit from variant specific boosters that are currently being developed. The CoVariant-SCAN is an ideal platform to identify those individuals because it could be conducted at the point-of-care, is easily manufactured at-scale, and can be deployed globally independent of a cold-chain or centralized testing laboratory.

### Example 5

### Demonstration of CoVariant-SCAN Modularity

Finally, to demonstrate the modular nature of the CoVariant-SCAN, the platform was adapted to detect nAbs against an additional VOC B.1.617.2 (also known as the Delta variant). B.1.617.2 contains two mutations within the RBD: L452R and T478K (FIG. 5A). The potency of the Regeneron therapeutic mAbs was examined on the assay against all VOCs. It was found that REGN10933, REGN10987 and the cocktail of both mAbs remained active against B.1.617.2 variant (FIG. 5B), which is consistent with previous studies. Next, plasma was tested from a new cohort of 19 individuals who received both doses of Pfizer (n=12), Moderna (n=4), or a single dose of the Johnson & Johnson vaccine (n=3) (FIG. 5C). The mean time since the first dose for this cohort was 15.9 weeks. It was found that there was a statistically significant difference in the percentage of ACE2-RBD blocking among the different VOCs as determined by a one-way ANOVA (F(4.90)=3.725, p=0.0075). ACE2 blocking by nAbs was lower for B.1.617.2 (p=0.026), P.1 (p=0.009) and B.1.351 (p=0.004) variants relative to WT, and there was no statistically significant difference for B.1.1.7 (p=0.196) as determined by Dunnett's multiple comparisons test. These findings are consistent with other studies, and suggest that the B.1.617.2 variant may be able to evade nAbs from vaccinee plasma; however, most individuals tested by the assay still developed some degree of neutralizing/blocking activity. This study highlights a key attribute of CoVariant-SCAN—the ability to rapidly incorporate additional RBD proteins from new VOCs as they emerge without the need to reoptimize the assay by simply adding another column of printed spots of the RBD for that VOC.

As shown here, a strength of the disclosed platform is the ability to rapidly test the impact of S protein mutations on immunity as they arise in newly emerging VOCs. The workflow only requires inkjet printing purified RBD proteins as a row of separate capture sites without any changes to the detection reagent, making further multiplexing simple. As SARS-CoV-2 variant sequences are identified and deposited into repositories, such as GISAID, recombinant RBDs from these variants can be quickly expressed, purified, and integrated into the assay, as demonstrated with the B.1.617.2 variant. Although mutations within the RBD were focused on, the full Si protein—which contains additional mutations in VOCs—can also be used as the pathogen on the CoVariant-SCAN. Likewise, the impact of modifications at important residues could prospectively assessed on immunity to identify mutations of concern. Therefore, the bespoke nature of CoVariant-SCAN can be useful to assess the impact of emerging SARS-CoV-2 mutations.

Overall, a rapid test was developed, termed the CoVariant-SCAN, to simultaneously assess the ability of antibodies to block the ACE2-RBD interaction against five SARS-CoV-2 variants: WT, B.1.1.7, P.1, B.1.351, and B.1.617.2. The assay is motivated by the urgent need for a rapid and easy-to-use assay that supplements conventional antibody neutralization tests which are labor-intensive, costly, require highly trained personnel, and are thus inaccessible in many regions around the world. While other assays have been developed that measure the ability of nAbs to block ACE2-RBD binding, it is believed that the CoVariant-SCAN is the first test that can detect nAbs against several SARS-CoV-2 variants simultaneously within 1 h. Furthermore, because the CoVariant-SCAN is built upon a “nonfouling” polymer coating which eliminates nearly all non-specific binding, the assay can be conducted directly from undiluted plasma.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the device structures, substituents, derivatives, intermediates, syntheses, compositions, formulations, or methods of use of the invention, may be made without departing from the spirit and scope thereof.

What is claimed is:

1. A method of detecting a neutralizing antibody, the method comprising:
contacting a biological sample with a device, the device comprising
a substrate;
a non-fouling layer positioned on the substrate, the non-fouling layer including a brush-like polymer;
a plurality of pathogen regions positioned on the non-fouling layer, each pathogen region including a different pathogen;
at least one detection region positioned on the non-fouling layer spatially separated from the pathogen regions, the detection region including a detection agent and an excipient, wherein the detection agent solubilizes upon contacting the biological sample and is capable of specifically binding to each pathogen; and
detecting the presence of a neutralizing antibody in the biological sample for each pathogen, wherein the presence of the neutralizing antibody is detected by inhibiting the binding of the detection agent to each pathogen.

2. The method of claim 1, wherein detecting the presence of the neutralizing antibody occurs in less than or equal to 1 hour after the biological sample contacts the device.

3. The method of claim 2, wherein detecting the presence of the neutralizing antibody occurs in less than or equal to 30 minutes after the biological sample contacts the device.

4. The method of claim 1, wherein each pathogen comprises a virus, a viral protein, or a combination thereof.

5. The method of claim 1, wherein each pathogen comprises a spike (S) protein or a variant thereof.

6. The method of claim 1, wherein each pathogen comprises a viral protein derived from SARS-CoV-2 or a variant thereof.

7. The method of claim 1, wherein the device comprises 2 to 20 pathogen regions, each pathogen region including a different pathogen.

8. The method of claim 1, wherein the device comprises a plurality of detection regions.

9. The method of claim 1, wherein the detection agent comprises a peptide, a protein, a carbohydrate, a lipid, a small molecule ligand, or a combination thereof.

10. The method of claim 1, wherein the detection agent comprises an extracellular receptor that is a pathological binding partner of each pathogen.

11. The method of claim 1, wherein the detection agent comprises angiotensin-converting enzyme 2 (ACE2) or a variant thereof.

12. The method of claim 1, wherein the detection agent comprises a detection moiety selected from the group consisting of a chromophore, a fluorophore, a radiolabel, a polynucleotide, a small molecule, an enzyme, a nanoparticle, a microparticle, a quantum dot, and an upconverter.

13. The method of claim 1, wherein the excipient comprises a salt, a carbohydrate, a polyol, an emulsifier, a water soluble polymer, or a combination thereof.

14. The method of claim 1, wherein the excipient comprises trehalose.

15. The method of claim 1, wherein the detection region further comprises heparin.

16. The method of claim 1, wherein the brush-like polymer comprises a monomer core group and a protein-resistant head group coupled to the monomer core group.

17. The method of claim 1, wherein the brush-like polymer comprises poly(oligo(ethylene glycol)methyl methacrylate) (POEGMA).

18. The method of claim 1, wherein the neutralizing antibody comprises an anti-SARS-CoV-2 antibody.

19. The method of claim 1, wherein the sample comprises blood, plasma, serum, or saliva.

20. The method of claim 1, wherein the substrate comprises a glass, a silicon, a metal oxide, a polymer, or a combination thereof.

21. A method of determining a neutralizing activity of a vaccine, the method comprising:
obtaining a biological sample from a subject that has received a vaccine;
contacting a biological sample with a device, the device comprising
a substrate;
a non-fouling layer positioned on the substrate, the non-fouling layer including a brush-like polymer;
a plurality of pathogen regions positioned on the non-fouling layer, each pathogen region including a different pathogen;
at least one detection region positioned on the non-fouling layer spatially separated from the pathogen regions, the detection region including a detection agent and an excipient, wherein the detection agent solubilizes upon contacting the biological sample and is capable of specifically binding to each pathogen; and
detecting the presence of a neutralizing antibody induced by the vaccine for each pathogen, wherein the presence of the neutralizing antibody is detected by inhibiting the binding of the detection agent to each pathogen.

22. The method of claim 21, wherein the vaccine comprises a vaccine against a pathogen that specifically binds to an extracellular receptor binding partner.

23. The method of claim 21, wherein the vaccine comprises a vaccine against SARS-CoV-2.

24. The method of claim 21, wherein each pathogen corresponds to an individual virus or a variant thereof.

25. The method of claim 21, wherein the subject is human.
